(19) United States
(12) Patent Application Publication
(10) Pub. No.: US 2005/0014679 A1
(43) Pub. Date: Jan. 20, 2005

(54) Insulin molecule having protracted time action

(76) Inventors: John Michael Beals, Indianapolis, IN (US); Michael Rosario DeFelippis, Carmel, IN (US); Richard Dennis DiMarchi, Carmel, IN (US); Wayne David Kohn, Indianapolis, IN (US); Radmila Micanovic, Indianapolis, IN (US); Sharon Ruth Myers, Indianapolis, IN (US); Kingman Ng, Carmel, IN (US); Lianshan Zhang, Carmel, IN (US)

Correspondence Address: ELI LILLY AND COMPANY;PATENT DIVISION, P.O. BOX 6288, INDIANAPOLIS, IN, 46206-6288, US

(21) Appl. No.: 10/496,847

(22) PCT Filed: Dec. 12, 2002
(86) PCT No.: PCT/US02/37601

Related U.S. Application Data
(60) Provisional application No. 60/344,310, filed on Dec. 20, 2001.
(60) Provisional application No. 60/414,604, filed on Sep. 27, 2002.

(51) Int. Cl. A61K 38/28

(52) U.S. Cl. 514/3; 530/303

(57) ABSTRACT

The present invention provides an insulin molecule that provides a protracted, even basal duration of action. The insulin molecule comprises a modification at the N-terminus of the A-chain, optionally a modification at the N-terminus of the B-chain, a modification at a B-chain lysine, and optionally a modification at the C-terminus of the A-chain. The present invention also provides a method of treating diabetes mellitus comprising administering the insulin molecule.

This application claims priority benefit of U.S. provisional application No. 60/344,310, filed Dec. 20, 2001, and of U.S. provisional application No. 60/414,604, filed Sep. 27, 2002, which are incorporated by reference in their entireties.

### FIELD OF THE INVENTION

The present invention relates to insulin molecules that are useful for treating the hyperglycemia that is characteristic of diabetes mellitus.

### BACKGROUND OF THE INVENTION

The physiological demand for insulin can be separated into two phases: (a) the nutrient absorptive phase requiring a pulse of insulin to dispose of the meal-related blood glucose surge, and (b) the post-absorptive phase requiring a sustained delivery of insulin to regulate hepatic glucose output for maintaining optimal fasting blood glucose, also known as a “basal” insulin secretion.

Effective insulin therapy for people with diabetes generally involves the combined use of two types of exogenous insulin formulations: a rapid-acting, mealtime insulin provided by bolus injections, and a longer-acting insulin, administered by injection once or twice daily to control blood glucose levels between meals.

An ideal exogenous basal insulin would provide an extended and “flat” time action—that is, it would control blood glucose levels for at least 12 hours, and preferably for 24 hours, without significant risk of hypoglycemia.

Commercially used longer-acting insulin molecules do not provide an insulin effect for 24 hours. Accordingly, there remains a need for an insulin molecule that provides an insulin effect for up to 24 hours.

### SUMMARY OF THE INVENTION

The present invention provides an insulin molecule having

(a) an A-chain of Formula I,

A-1 A0 A1 A2 A3 A4 A5 A6 A7 A8 A9 A10
Xaa-Xaa-Gly-Ile-Val-Glu-Gln-Cys-Cys-Thr-Ser-Ile-
A11 A12 A13 A14 A15 A16 A17 A18 A19 A20 A21
Cys-Ser-Leu-Tyr-Gln-Leu-Glu-Asn-Tyr-Cys-Xaa,

wherein the amino acid sequence of Formula I is set forth in Seq. ID No. 1, and (b) a B-chain of Formula II,

B-1 B0 B1 B2 B3 B4 B5 B6 B7 B8 B9 B10
Xaa-Xaa-Phe-Val-Asn-Gln-His-Leu-Cys-Gly-Ser-His-
B11 B12 B13 B14 B15 B16 B17 B18 B19 B20 B21 B22
Leu-Val-Glu-Ala-Leu-Tyr-Leu-Val-Cys-Gly-Glu-Arg-
B23 B24 B25 B26 B27 B28 B29 B30
Gly-Phe-Phe-Tyr-Thr-Xaa-Xaa-Xaa,

wherein the amino acid sequence of Formula II is set forth in Seq. ID No. 2, wherein
Xaa at position A-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position A0 is Arg, derivatized Arg, homoarginine, desamino homoarginine, desaminoarginine, Lys, derivatized Lys, desaminolysine, alpha guanidino homoarginine, or alpha methyl arginine;
Xaa at position A21 is a genetically encodable amino acid;
Xaa at position B-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position B0 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position B28 is Lys or Pro;
Xaa at position B29 is Lys or Pro;
Xaa at position B30 is Thr, Ala or is absent;
one of Xaa at position B28 or Xaa at position B29 is Lys;
Xaa at position B28 and Xaa at position B29 are not both Lys; and
the ε-amino group of Lys at position B28 or B29 is covalently bound to the α-carboxyl group of a positively charged amino acid to form a Lys-Nε-aminoacid derivative.

The present invention also provides a method of treating diabetes mellitus, the method comprising administering to a subject the insulin molecule of the present invention in an amount sufficient to regulate blood glucose concentration.

The present invention also provides microcrystals comprising the insulin molecule of the present invention, methods of making the microcrystals, and a method of treating diabetes by administering the microcrystals.

The present invention also provides a suspension formulation comprising an insoluble phase and a solution phase, the insoluble phase comprising the microcrystal of the present invention, and the solution phase comprising water. The present invention also provides a method of making the suspension formulation.

The present invention also provides a method of treating diabetes mellitus, the method comprising administering the suspension formulation to a subject in an amount sufficient to regulate blood glucose concentration in the subject.

The present invention also provides a process for preparing the suspension formulation. The present invention also provides a method of treating diabetes mellitus, the method comprising administering the suspension formulation to a subject in an amount sufficient to regulate blood glucose concentration in the subject.

The present invention also provides a method of making an insulin molecule, comprising: (a) acylating each free amino group of an insulin template with a protected amino acid or protected amino acid derivative to form an acylated insulin molecule; (b) purifying the acylated insulin molecule; (c) removing the protecting group from each protected amino acid or protected amino acid derivative to form a deprotected acylated insulin molecule; and (d) purifying the deprotected acylated insulin molecule. In one preferred embodiment, the protected amino acid is protected Arg, and the amino acid is Arg. In another preferred embodiment, the protected amino acid is protected Lys; and the amino acid is Lys.

### BRIEF DESCRIPTION OF THE FIGURE

FIG. 1 depicts the Lys-Nε-Arg derivative obtained by forming a covalent bond between the ε-amino group of Lys and the α-carboxyl group of Arg.

### DETAILED DESCRIPTION OF THE INVENTION

In one preferred embodiment, the present invention provides an insulin molecule comprising a modification at one or more of the N-terminus of the insulin A-chain, the C-terminus of the insulin A-chain, the N-terminus of the insulin B-chain, and a B-chain lysine.

In another preferred embodiment, the insulin molecule of the present invention comprises a modification of the N-terminus of the A-chain, a modification of the N-terminus of the B-chain, a modification of a B-chain lysine, and optionally a modification of the C-terminus of the A-chain. For example, such an insulin molecule is one in which an Arg has been covalently attached to the N-terminus of the A-chain, an Arg has been covalently attached to the N-terminus of the B-chain, a B-chain Lys has been modified, and optionally the C-terminal amino acid of the A-chain has been substituted with another amino acid, such as Gly.

In another preferred embodiment, the insulin molecule of the present invention comprises a modification of the N-terminus of the A-chain, a modification of a B-chain lysine, and optionally a modification of the C-terminus of the A-chain. For example, such an insulin molecule is one in which an Arg has been covalently attached to the N-terminus of the A-chain, a B-chain Lys has been modified, and optionally the C-terminal amino acid of the A-chain has been substituted with another amino acid, such as Gly.

In another preferred embodiment, the present invention provides an insulin molecule having

(a) an A-chain of Formula I,

A-1 A0 A1 A2 A3 A4 A5 A6 A7 A8 A9 A10
Xaa-Xaa-Gly-Ile-Val-Glu-Gln-Cys-Cys-Thr-Ser-Ile-
A11 A12 A13 A14 A15 A16 A17 A18 A19 A20 A21
Cys-Ser-Leu-Tyr-Gln-Leu-Glu-Asn-Tyr-Cys-Xaa,

wherein the amino acid sequence of Formula I is set forth in Seq. ID No. 1, and

(b) a B-chain of Formula II,

B-1 B0 B1 B2 B3 B4 B5 B6 B7 B8 B9 B10
Xaa-Xaa-Phe-Val-Asn-Gln-His-Leu-Cys-Gly-Ser-His-
B11 B12 B13 B14 B15 B16 B17 B18 B19 B20 B21 B22
Leu-Val-Glu-Ala-Leu-Tyr-Leu-Val-Cys-Gly-Glu-Arg-
B23 B24 B25 B26 B27 B28 B29 B30
Gly-Phe-Phe-Tyr-Thr-Xaa-Xaa-Xaa,

wherein the amino acid sequence of Formula II is set forth in Seq. ID No. 2, wherein the amino acid sequence of Formula II is set forth in Seq. ID No. 2, wherein
Xaa at position A-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position A0 is Arg, derivatized Arg, homoarginine, desamino homoarginine, desaminoarginine, Lys, derivatized Lys, desaminolysine, alpha guanidino homoarginine, or alpha methyl arginine;
Xaa at position A21 is a genetically encodable amino acid;
Xaa at position B-1 is Arg, derivatized Arg, homoarginine, desamino homoarginine, desaminoarginine, Lys, derivatized Lys, desaminolysine, alpha guanidine homoarginine, alpha methyl arginine, or is absent;
Xaa at position B0 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position B28 is Lys or Pro;
Xaa at position B29 is Lys or Pro;
Xaa at position B30 is Thr, Ala or is absent;
one of Xaa at position B28 or Xaa at position B29 is Lys;
Xaa at position B28 and Xaa at position B29 are not both Lys; and

the ε-amino group of Lys at position B28 or B29 is covalently bound to the α-carboxyl group of a positively charged amino acid.

In one preferred embodiment, Xaa at position A-1 is absent, Xaa at position A0 is Arg, derivatized Arg, desaminoarginine, Lys, derivatized Lys, alpha guanidino homoarginine, or alpha methyl arginine, Xaa at position B-1 is absent, and Xaa at position B0 is Arg, derivatized Arg, desaminoarginine, Lys, derivatized Lys, alpha guanidino homoarginine, alpha methyl arginine, or is absent.

In another preferred embodiment, Xaa at position A-1 is absent, Xaa at position A0 is Arg, Xaa at position B-1 is absent, and Xaa at position B0 is absent.

In another preferred embodiment, Xaa at position A-1 is absent, Xaa at position A0 is derivatized Lys, Xaa at position B-1 is absent, and Xaa at position B0 is absent.

“Formula I” is

A-1 A0 A1 A2 A3 A4 A5 A6 A7 A8 A9 A10
Xaa-Xaa-Gly-Ile-Val-Glu-Gln-Cys-Cys-Thr-Ser-Ile-
A11 A12 A13 A14 A15 A16 A17 A18 A19 A20 A21
Cys-Ser-Leu-Tyr-Gln-Leu-Glu-Asn-Tyr-Cys-Xaa,

and the amino acid sequence of Formula I is set forth in Seq. ID No. 1. The amino acids at positions A-1 to A21 of Formula I correspond, respectively, to the amino acids at positions 1-23 of Seq. ID No. 1. The amino acids at positions A1 to A20 of Formula I and at positions 3-22 of Seq. ID No. 1 correspond to the amino acids at positions 1-20 of the A-chain of human insulin (Seq. ID No. 3).

“Formula II” is

B-1 B0 B1 B2 B3 B4 B5 B6 B7 B8 B9 B10
Xaa-Xaa-Phe-Val-Asn-Gln-His-Leu-Cys-Gly-Ser-His-
B11 B12 B13 B14 B15 B16 B17 B18 B19 B20 B21 B22
Leu-Val-Glu-Ala-Leu-Tyr-Leu-Val-Cys-Gly-Glu-Arg-
B23 B24 B25 B26 B27 B28 B29 B30
Gly-Phe-Phe-Tyr-Thr-Xaa-Xaa-Xaa,

and the amino acid sequence of Formula II is set forth in Seq. ID No. 2. The amino acids at positions B-1 to B30 of Formula II correspond, respectively, to the amino acids at positions 1-32 of Seq. ID No. 2. The amino acids at positions B1 to B27 of Formula II and at positions 3-29 of Seq. ID No. 2 correspond to the amino acids at positions 1-27 of the B-chain of human insulin (Seq. ID No. 4).

Polynucleotide and amino acid sequences of insulin molecules from a number of different species are well known to those of ordinary skill in the art. Preferably, “insulin” means human insulin. “Human insulin” has a twenty-one amino acid A-chain, which is Gly-Ile-Val-Glu-Gln-Cys-Cys-Thr-Ser-Ile-Cys-Ser-Leu-Tyr-Gln-Leu-Glu-Asn-Tyr-Cys-Asn (Seq. ID No. 3), and a thirty-amino acid B-chain, which is Phe-Val-Asn-Gin-His-Leu-Cys-Gly-Ser-His-Leu-Val-Glu-Ala-Leu-Tyr-Leu-Val-Cys-Gly-Glu-Arg-Gly-Phe-Phe-Tyr-Thr-Pro-Lys-Thr (Seq. ID No. 4).

The A- and B-chains in human insulin are cross-linked by disulfide bonds. One interchain disulfide bond is between the Cys at position A7 of Formula I and the Cys at position B7 of Formula II, and the other interchain disulfide bond is between the Cys at position A20 of Formula I and the Cys at position B139 of Formula II. An intrachain disulfide bond is between the Cysteines at positions A6 and A11 of Formula I.

The terms “a host cell” and “the host cell” refer to both a single host cell and to more than one host cell.

“Insulin molecule” as used herein encompasses wild-type insulins, insulin derivatives, and insulin analogs.

“Positively charged amino acid” is a natural or non-natural amino acid that has a net positive charge at pH 6.0. In one preferred embodiment, the positively charged amino acid is Arg. In another preferred embodiment, the positively charged amino acid is Lys.

“Insulin derivative” as used herein means an insulin molecule in which a Lys is derivatized to form a covalent bond between the ε-amino group ('Nε) of a Lys and another moiety. In one preferred embodiment, an A-chain Lys is derivatized to form a covalent bond between the ε-amino group of a Lys and another moiety. In another preferred embodiment, a B-chain Lys is derivatized to form a covalent bond between the ε-amino group group of a Lys and another moiety. In another preferred embodiment, both an A-chain Lys and a B-chain Lys are derivatized to form a covalent bond between the ε-amino group group of each Lys and another moiety.

In another preferred embodiment, the covalent bond is formed by acylation with a positively charged amino acid. In this embodiment, a covalent bond is formed between the ε-amino group of a Lys and the carbon in the α-carboxyl group of an amino acid when a hydrogen atom from the ε-amino group of Lys and the hydroxyl portion of the α-carboxyl group of an amino acid leave and form water upon the covalent bonding of the amino acid to Lys to form a covalent bond.

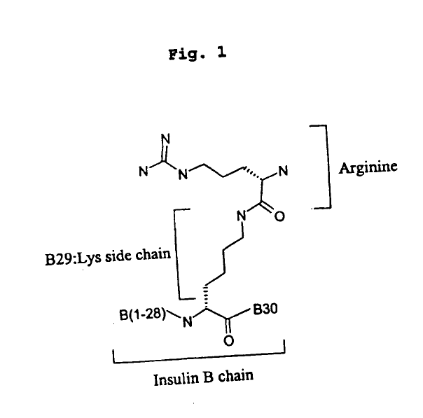

In another preferred embodiment, a covalent bond is formed between the ε-amino group of a Lys and the carbon in the α-carboxyl group of Arg, forming the “Lys-Nε-Arg” derivative. The Lys-Nε-Arg derivative is shown in FIG. 1. In another preferred embodiment, the Lys-Nε-Arg insulin derivative is formed from a Lys at position B28 of Formula II. In another preferred embodiment, the Lys-N^{ε}-Arg insulin derivative is formed from a Lys at position B29 of Formula II, which corresponds to the Lys at position 29 of Seq. ID No. 4.

In another preferred embodiment, a covalent bond is formed between the ε-amino group of a Lys and the carbon in the α-carboxyl group of Lys, forming the “Lys-Nε-Lys” derivative. In another preferred embodiment, the Lys-Nε-Lys insulin derivative is formed from a Lys at position B28 of Formula II. In another preferred embodiment, the Lys-Nε-Lys insulin derivative is formed from a Lys at position B29 of Formula II, which corresponds to the Lys at position 29 of Seq. ID No. 4.

“Proinsulin derivative” as used herein means a proinsulin molecule in which a Lys is derivatized to form a covalent bond between tile ε-amino group of a Lys and another moiety. In one preferred embodiment, the covalent bond is formed by acylation with a positively charged amino acid. In this embodiment, a covalent bond is formed between the ε-amino group of a Lys and the carbon in the α-carboxyl group of a positively charged amino acid, forming the “Lys-Nε-amino acid” derivative. In one preferred embodiment, a covalent bond is formed between the ε-amino group of a Lys and the carbon in the α-carboxyl group of Arg, forming the “Lys-N□-Arg” derivative. In another preferred embodiment, the Lys-Nε-Arg insulin derivative is formed from a Lys at position B28 of Formula II. In another preferred embodiment, the Lys-Nε-Arg insulin derivative is formed from a Lys at position B29 of Formula II, which corresponds to the Lys at position 29 of Seq. ID No. 4. In another preferred embodiment, a covalent bond is formed between the ε-amino group of a Lys and the carbon in the α-carboxyl group of Lys, forming the “Lys-Nε-Lys” derivative. In another preferred embodiment, the Lys-Nε-Lys insulin derivative is formed from a Lys at position B28 of Formula II. In another preferred embodiment, the Lys-Nε-Lys insulin derivative is formed from a Lys at position B29 of Formula II, which corresponds to the Lys at position 29 of Seq. ID No. 4.

“Insulin analog” as used herein is different from an “insulin derivative” as used herein. An “insulin derivative” is an insulin molecule in which a Lys is derivatized to form a covalent bond between the ε-amino group of Lys and another moiety. In contrast to an “insulin derivative,” an “insulin analog” is an insulin molecule that is modified to differ from a wild-type insulin, but a Lys is not derivatized to form a covalent bond between the ε-amino group of Lys and another moiety. Thus, an insulin analog can have A- and/or B-chains that have substantially the same amino acid sequences as the A-chain and the B-chain of human insulin, respectively, but differ from the A-chain and B-chain of human insulin by having one or more amino acid deletions in the A- and/or B-chains, and/or one or more amino acid replacements in the A- and/or B-chains, and/or one or more amino acids covalently bound to the N— and/or C-termini of the A-and/or B-chains.

Thus, for example, A0^{Arg}B29^{Lys-Nε-Arg}-insulin and A0^{Lys-Nε-Arg}-insulin and A0^{Lys-Nε-Arg}B29^{Lys-Nε-Arg} insulin are insulin derivatives, because in each of those molecules, a Lys is derivatized to form a covalent bond between the ε-amino group of Lys and another moiety (Arg). In contrast to those insulin derivatives, A0^{Arg}-insulin is an insulin analog, because in A0^{Arg}-insulin, a Lys is not derivatized to form a covalent bond between the ε-amino group of Lys and another moiety.

“Proinsulin analog” as used herein is different from a “proinsulin derivative” as used herein. A “proinsulin derivative” is a proinsulin molecule in which a Lys is derivatized to form a covalent bond between the ε-amino group of a Lys and another moiety. In contrast to a “proinsulin derivative,” a “proinsulin analog” is a proinsulin molecule that is modified to differ from a wild-type proinsulin, but a Lys is not derivatized to form a covalent bond between the ε-amino group of Lys and another moiety.

Thus, a proinsulin analog can have an A-chain, a B-chain and/or a C-peptide that have substantially the same amino acid sequences as the A-chain, B-chain and C-peptide in human proinsulin, respectively, but differ from the A-chain, B-chain and C-peptide of human proinsulin by having one or more amino acid deletions in the A-chain, B-chain or C-peptide, and/or one or more amino acid replacements in the A-chain, B-chain or C-peptide, and/or one or more amino acids covalently bound to the N— and/or C-termini of the A-chain, B-chain or C-peptide. For example, A0^{Arg}B29^{Lys-Nε-Arg}-proinsulin is an insulin derivative, but B28^{Lys}B29^{Pro}-proinsulin is a proinsulin analog.

The amino acid at the Xaa at position A-1 of Formula I can be present or absent. If it is present, it is preferably Arg, derivatized Arg, homoarginine, desamino homoarginine, desaminoarginine, Lys, derivatized Lys, desaminolysine, alpha guanidino homoarginine, or alpha methyl arginine.

The amino acid at the Xaa at position A0 must be present. In a preferred embodiment, Xaa at position A0 is Arg, derivatized Arg, homoarginine, desamino homoarginine, desaminoarginine, Lys, derivatized Lys, desaminolysine, alpha guanidino homoarginine, or alpha methyl arginine. In a preferred embodiment, the Xaa at position A0 is Lys derivatized with a positively charged amino acid. In another preferred embodiment, the Xaa at position A0 is Lys-Nε-Arg. In another preferred embodiment, the Xaa at position A0 is Lys-Nε-Lys.

The amino acid at the Xaa at position A21 is a genetically encodable amino acid selected from the group consisting of alanine (Ala), arginine (Arg), asparagine (Asn), aspartic acid (Asp), cysteine (Cys), glutamatic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val). In one preferred embodiment, the amino acid at the Xaa at position A21 is glycine. In another preferred preferred embodiment, the amino acid at the Xaa at position A21 is serine. In another preferred embodiment, the amino acid at the Xaa at position A21 is threonine. In another preferred embodiment, the amino acid at the Xaa at position A21 is alanine.

The amino acid at the Xaa at position B-1 of Formula II can be present or absent. If it is present, it is preferably Arg, derivatized Arg, homoarginine, desamino homoarginine, desaminoarginine, Lys, derivatized Lys, desaminolysine, alpha guanidino homoarginine, or alpha methyl arginine. The amino acid at the Xaa at position B0 can be present or absent. If it is present, it is preferably Arg, derivatized Arg, homoarginine, desamino homoarginine, desaminoarginine, Lys, derivatized Lys, desaminolysine, alpha guanidino homoarginine, or alpha methyl arginine. If the amino acid at the Xaa at position B0 is absent, then the amino acid at the Xaa at position B-1 is also absent.

The amino acid at the Xaa at position B28 is Lys or Pro.

The amino acid at the Xaa at position B29 is Lys or Pro.

The amino acid at the Xaa at position B30 is Thr, Ala or is absent.

In one preferred embodiment, either the Xaa at position B28 or the Xaa at position B29 is Lys, but the Xaa at position B28 and the Xaa at position B29 are not both Lys, and the ε-amino group of the Lys at position B28 or B29 is covalently bound to the ε-carboxyl group of a positively charged amino acid to form the Lys-Nε-amino acid derivative. In another preferred embodiment, the Ε-amino group of the Lys at position B28 or B29 is covalently bound to the ε-carboxyl group of Arg to form the Lys-Nε-Arg derivative. In another preferred embodiment, the ε-amino group of the Lys at position B28 or B29 is covalently bound to the F-carboxyl group of Lys to form the Lys-Nε-Lys derivative.

In another preferred embodiment, an amino acid in an insulin molecule is further derivatized. In one preferred embodiment, the amino acid derivatization is acylation. More preferably, Lys at position B29 of Formula II is acylated with an amino acid.

In another preferred embodiment, the amino acid derivatization is carbamylation. Preferably, a Lys is derivatized to form homoarginne. More preferably, homoarginine is formed from Lys at position B29 of Formula II.

“Polypeptide chain” means two or more amino acids linked together via peptide bonds.

In a preferred embodiment, the A-chain of the insulin molecule of the present invention is crosslinked to the B-chain via two disulfide bonds, and the A-chain contains an intrachain disulfide bond crosslinkage. More specifically, “properly linked” means (1) a disulfide bond between the Cys at position A6 of Formula I and the Cys at position A11, (2) a disulfide bond between the Cys at position A7 of Formula I and the Cys at position B7 of Formula II, and (3) a disulfide bond between the Cys at position A20 of Formula I and the Cys at position B19 of Formula II.

A simple shorthand notation is used herein to denote insulin and proinsulin molecules, and is set forth with reference to the A-chain of Formula I (Seq. ID No. 1) and the B-chain of Formula II (Seq. ID No. 2). In this notation, if an amino acid at the Xaa at position A-1, B-1 or B0 is not mentioned in the shorthand name of an insulin molecule, then the Xaa at that position is absent. If an amino acid at the Xaa at position A21 is not mentioned in the shorthand name of an insulin molecule, then the amino acid is Asn, which is the amino acid at position A21 in the wild-type insulin A-chain (Seq. ID No. 3). If an amino acid at the Xaa at position B28 is not mentioned in the shorthand name of an insulin molecule, then the amino acid is Pro, which is the amino acid at position B28 in the wild-type insulin B-chain (Seq. ID No. 4). If an amino acid at the Xaa at position B29 is not mentioned in the shorthand name of an insulin molecule, then the amino acid is Lys, which is the amino acid at position B29 in the wild-type insulin B-chain. If an amino acid at the Xaa at position B30 is not mentioned in the shorthand name of an insulin molecule, then the amino acid is Thr, which is the amino acid at position B30 in the wild-type insulin B-chain. “des(B30)” means that the Xaa at position B30 is absent. If an amino acid in a proinsulin is not mentioned in the shorthand name of a proinsulin molecule, the amino acid at that position is the amino acid at that position in the wild-type human proinsulin molecule.

A non-limiting example of the shorthand notation is “A0^{Arg}A21^{Xaa}B0^{Arg}B29^{Lys-Nε-Arg}-insulin,” which means that the Xaa at position A-1 of Formula I is absent, the Xaa at position A0 is Arg, the Xaa at position A21 is a genetically encodable amino acid, the Xaa at position B-1 of Formula II is absent, the Xaa at position B0 is Arg, the Xaa at position B28 is Pro, the Xaa at position B29 is Lys-Nε-Arg, and the Xaa at position B30 is Thr.

In another non-limiting example of the shorthand notation, the shorthand notation “A0^{Lys-Nε-Arg}A21^{Gly}B29^{Lys-Nε-Arg}-insulin” means that the Xaa at position A-1 of Formula I is absent, the Xaa at position A0 is Lys-Nε-Arg, the Xaa at position A21 is Gly, the Xaa at position B-1 of Formula II is absent, the Xaa at position B0 is absent, the Xaa at position B28 is Pro, the Xaa at position B29 is Lys-Nε-Arg, and the Xaa at position B30 of is Thr.

In another non-limiting example of the shorthand notation, the shorthand notation for “A21^{Xaa}-insulin” means that the Xaa at position A-1 of Formula I is absent, the Xaa at positions A0 is absent, the Xaa at position A21 is a genetically encodable amino acid, the Xaa at position B-1 of Formula II is absent, the Xaa at position B0 is absent, the Xaa at position B28 is Pro, the Xaa at position B29 is Lys, and the Xaa at position B30 is Thr. “A21^{Gly}-insulin” is the same as A21^{Xaa}-insulin, except that the Xaa at position A21 is Gly. A21^{Ser}-insulin” is the same as A21^{Xaa}-insulin, except that the Xaa at position A21 is Ser.

In another non-limiting example of the shorthand notation, the shorthand notation for “B₂₈^{Lys}B29^{Pro}-insulin” means that the Xaa at position A-1 of Formula I is absent, the Xaa at positions A0 is absent, the Xaa at position A21 is a genetically encodable amino acid, the Xaa at position B-1 of Formula II is absent, the Xaa at position B0 is absent, the Xaa at position B28 is Lys, the Xaa at position B29 is Pro, and the Xaa at position B30 is Thr.

In another non-limiting example of the shorthand notation, the shorthand notation “A0^{Arg}-insulin means that the Xaa at position A-1 of Formula I is absent, the Xaa at position A0 is Arg, the Xaa at position A21 is Asn, the Xaa at position B-1 of Formula II is absent, the Xaa at position B0 is absent, the Xaa at position B28 is Pro, the Xaa at position B29 is Lys, and the Xaa at position B30 of is Thr. See U.S. Pat. No. 5,506,202, and U.S. Pat. No. 5,430,016.

“gHR” means alpha-guanidyl homoarginine.

In a preferred embodiment, the insulin molecule of the present invention is selected from the group consisting of:
A0^{Arg}B29^{Lys-Nε-Arg}-insulin;
A0^{Arg}A21^{Xaa}B29^{Lys-Nε-Arg}-insulin;
A0^{Arg}A21^{Gly}B29^{Lys-Nε-Arg}-insulin;
A0^{Arg}A21^{Ser}B29^{Lys-Nε-Arg}-insulin;
A0^{Arg}B29^{Lys-Nε-Lys}-insulin;
A0^{Arg}A21^{Xaa}B29^{Lys-Nε-Lys}-insulin;
A0^{Arg}A21^{Gly}B29^{Lys-Nε-Lys}-insulin;
A0^{Arg}A21^{Ser}B29^{Lys-Nε-Lys}-insulin;
A0^{Lys}B29^{Lys-Nε-Arg}-insulin;
A0^{Lys}A21^{Xaa}B29^{Lys-Nε-Arg}-insulin;
A0^{Lys}A21^{Gly}B29^{Lys-Nε-Arg}-insulin;
A0^{Lys}A21^{Ser}B29^{Lys-Nε-Arg}-insulin;
A0^{Lys}B29^{Lys-Nε-Lys}-insulin;
A0^{Lys}A21^{Xaa}B29^{Lys-Nε-Lys}-insulin;
A0^{Lys}A21^{Gly}B29^{Lys-Nε-Lys}-insulin;
A0^{Lys}A21^{Ser}B29^{Lys-Nε-Lys}-insulin;
A-1^{Arg}A0^{Lys}A21^{Xaa}B29^{Lys-Nε-Arg}-insulin;
A-1^{Arg}A0^{Lys}A21^{Gly}B29^{Lys-Nε-Arg}-insulin;
A-1^{Arg}A0^{Lys}A21^{Ser}B29^{Lys-Nε-Arg}-insulin;
A-1^{Arg}A0^{Lys}A21^{Xaa}B29^{Lys-Nε-Lys}-insulin;
A-1^{Arg}A0^{Lys}A21^{Gly}B29^{Lys-Nε-Lys}-insulin;
A-1^{Arg}A0^{Lys}A21^{Ser}B29^{Lys-Nε-Lys}-insulin;
A-1^{Lys}A0^{Lys}A21^{Xaa}B29^{Lys-Nε-Arg}-insulin;
A-1^{Lys}A0^{Lys}A21^{Gly}B29^{Lys-Nε-Arg}-insulin;
A-1^{Lys}A0^{Lys}A21^{Ser}B29^{Lys-Nε-Arg}-insulin;
A-1^{Lys}A0^{Lys}A21^{Xaa}B29^{Lys-Nε-Lys}-insulin;
A-1^{Lys}A0^{Lys}A21^{Gly}B29^{Lys-Nε-Lys}-insulin;
A-1^{Lys}A0^{Lys}A21^{Ser}B29^{Lys-Nε-Lys}-insulin;
A-1^{Arg}A0^{Arg}A21^{Xaa}B29^{Lys-Nε-Arg}-insulin;
A-1^{Arg}A0^{Arg}A21^{Gly}B29^{Lys-Nε-Arg}-insulin;
A-1^{Arg}A0^{Arg}A21^{Ser}B29^{Lys-Nε-Arg}-insulin;
A-1^{Arg}A0^{Arg}A21^{Xaa}B29^{Lys-Nε-Lys}-insulin;
A-1^{Arg}A0^{Arg}A21^{Gly}B29^{Lys-Nε-Lys}-insulin;
A-1^{Arg}A0^{Arg}A21^{Ser}B29^{Lys-Nε-Lys}-insulin;
A0^{Lys-Nε-Arg}A21^{Xaa}B29^{Lys-Nε-Arg}-insulin;
A0^{Lys-Nε-Arg}A21^{Gly}B29^{Lys-Nε-Arg}-insulin;
A0^{Lys-Nε-Arg}A21^{Ser}B29^{Lys-Nε-Arg}-insulin;
A0^{Lys-Nε-Arg}A21^{Xaa}B29^{Lys-Nε-Lys}-insulin;
A0^{Lys-Nε-Arg}A21^{Gly}B29^{Lys-Nε-Lys}-insulin;
A0^{Lys-Nε-Arg}A21^{Ser}B29^{Lys-Nε-Lys}-insulin;
A0^{Lys-Nε-Lys}A21^{Xaa}B29^{Lys-Nε-Arg}-insulin;
A0^{Lys-Nε-Lys}A21^{Gly}B29^{Lys-Nε-Arg}-insulin;
A0^{Lys-Nε-Lys}A21^{Ser}B29^{Lys-Nε-Arg}-insulin;
A0^{Lys-Nε-Lys}A21^{Xaa}B29^{Lys-Nε-Lys}-insulin;
A0^{Lys-Nε-Lys}A21^{Gly}B29^{Lys-Nε-Lys}-insulin;
A0^{Lys-Nε-Lys}A21^{Ser}B29^{Lys-Nε-Lys}-insulin;
A0^{Arg}B0^{Arg}B29^{Lys-Nε-Arg}-insulin;
A0^{Arg}A21^{Xaa}B0^{Arg}B29^{Lys-Nε-Arg}-insulin;
A0^{Arg}A21^{Gly}B0^{Arg}B29^{Lys-Nε-Arg}-insulin;
A0^{Arg}A21^{Ser}B0^{Arg}B29^{Lys-Nε-Arg}-insulin;
A0^{Arg}B0^{Arg}B29^{Lys-Nε-Lys}-insulin;
A0^{Arg}A21^{Xaa}B0^{Arg}B29^{Lys-Nε-Lys}-insulin;
A0^{Arg}A21^{Gly}B0^{Arg}B29^{Lys-Nε-Lys}-insulin;
A0^{Arg}A21^{Ser}B0^{Arg}B29^{Lys-Nε-Lys}-insulin;
A0^{Lys}B0^{Lys}B29^{Lys-Nε-Arg}-insulin;
A0^{Lys}A21^{Xaa}B0^{Lys}B29^{Lys-Nε-Arg}-insulin;
A0^{Lys}A21^{Gly}B0^{Lys}B29^{Lys-Nε-Arg}-insulin;
A0^{Lys}A21^{Ser}B0^{Lys}B29^{Lys-Nε-Arg}-insulin;
A0^{Lys}B0^{Lys}B29^{Lys-Nε-Lys}-insulin;
A0^{Lys}A21^{Xaa}B0^{Lys}B29^{Lys-Nε-Lys}-insulin;
A0^{Lys}A21^{Gly}B0^{Lys}B29^{Lys-Nε-Lys}-insulin;
A0^{Lys}A21^{Ser}B0^{Lys}B29^{Lys-Nε-Lys}-insulin;
A0^{Arg}B0^{Lys}B29^{Lys-Nε-Arg}-insulin;
A0^{Arg}A21^{Xaa}B0^{Lys}B29^{Lys-Nε-Arg}-insulin;
A0^{Arg}A21^{Gly}B0^{Lys}B29^{Lys-Nε-Arg}-insulin;
A0^{Arg}A21^{Ser}B0^{Lys}B29^{Lys-Nε-Arg}-insulin;
A0^{Lys}B0^{Arg}B29^{Lys-Nε-Arg}-insulin;
A0^{Lys}A21^{Xaa}B0^{Arg}B29^{Lys-Nε-Arg}-insulin;
A0^{Lys}A21^{Gly}B0^{Arg}B29^{Lys-Nε-Arg}-insulin;
A0^{Lys}A21^{Ser}B0^{Arg}B29^{Lys-Nε-Arg}-insulin;
A0^{Lys}B0^{Arg}B29^{Lys-Nε-Lys}-insulin;
A0^{Lys}A21^{Xaa}B0^{Arg}B29^{Lys-Nε-Lys}-insulin;
A0^{Lys}A21^{Gly}B0^{Arg}B29^{Lys-Nε-Lys}-insulin;
A0^{Lys}A21^{Ser}B0^{Arg}B29^{Lys-Nε-Lys}-insulin;
A0^{Arg}B0^{Lys}B29^{Lys-Nε-Lys}-insulin;
A0^{Arg}A21^{Xaa}B0^{Lys}B29^{Lys-Nε-Lys}-insulin;
A0^{Arg}A21^{Gly}B0^{Lys}B29^{Lys-Nε-Lys}-insulin;
A0^{Arg}A21^{Ser}B0^{Lys}B29^{Lys-Nε-Lys}-insulin;
A0^{gHR}B0^{gHR}B29^{Lys-Nε-Arg}-insulin;
A0^{gHR}A21^{Xaa}B0^{gHR}B29^{Lys-Nε-Arg}-insulin;
A0^{gHR}A21^{Gly}B0^{gHR}B29^{Lys-Nε-Arg}-insulin;
A0^{gHR}A21^{Ser}B0^{gHR}B29^{Lys-Nε-Arg}-insulin;
A0^{gHR}B0^{gHR}B29^{Lys-Nε-Lys}-insulin;
A0^{gHR}A21^{Xaa}B0^{gHR}B29^{Lys-Nε-Lys}-insulin;
A0^{gHR}A21^{Gly}B0^{gHR}B29^{Lys-Nε-Lys}-insulin;
A0^{gHR}A21^{Ser}B0^{gHR}B29^{Lys-Nε-Lys}-insulin;
A0^{Arg}A21^{Xaa}B0^{Arg}B28^{Lys-Nε-Arg}B29^{Pro}-insulin;
A0^{Arg}A21^{Xaa}B0^{Lys}B28^{Lys-Nε-Arg}B29^{Pro}-insulin;
A0^{Lys}A21^{Xaa}B0^{Arg}B28^{Lys-Nε-Arg}B29^{Pro}-insulin;
A0^{Lys}A21^{Xaa}B0^{Lys}B28^{Lys-Nε-Arg}B29^{Pro}-insulin;
A0^{Arg}A21^{Xaa}B28^{Lys-Nε-Arg}B29^{Pro}-insulin;
A0^{Arg}A21^{Gly}B28^{Lys-Nε-Arg}B29^{Pro}-insulin;
A0^{Arg}A21^{Ser}B28^{Lys-Nε-Arg}B29^{Pro}-insulin;
A0^{Arg}A21^{Xaa}B28^{Lys-Nε-Lys}B29^{Pro}-insulin;
A0^{Arg}A21^{Gly}B28^{Lys-Nε-Lys}B29^{Pro}-insulin;
A0^{Arg}A21^{Ser}B28^{Lys-Bε-Lys}B29^{Pro}-insulin;
A0^{Lys}A21^{Xaa}B28^{Lys-Nε-Arg}B29^{Pro}-insulin;
A0^{Lys}A21^{Gly}B28^{Lys-Nε-Arg}B29^{Pro}-insulin;
A0^{Lys}A21^{Ser}B28^{Lys-Nε-Arg}B29^{Pro}-insulin;
A0^{Lys}A21^{Xaa}B28^{Lys-Nε-Lys}B29^{Pro}-insulin;
A0^{Lys}A21^{Gly}B28^{Lys-Nε-Lys}B29^{Pro}-insulin; and
A0^{Lys}A21^{Ser}B28^{Lys-Nε-Lys}B29^{Pro}-insulin.
A0^{Arg}B29^{Lys-Nε-Arg}B31^{Arg}-insulin;
A0^{Arg}A21^{Xaa}B29^{Lys-Nε-Arg}B31^{Arg}-insulin;
A0^{Arg}A21^{Gly}B29^{Lys-Nε-Arg}B31^{Arg}-insulin;
A0^{Arg}A21^{Ser}B29^{Lys-Nε-Arg}B31^{Arg}-insulin;
A0^{Arg}B29^{Lys-Nε-Lys}B31^{Arg}-insulin;
A0^{Arg}A21^{Xaa}B29^{Lys-Nε-Lys}B31^{Arg}-insulin;
A0^{Arg}A21^{Gly}B29^{Lys-Nε-Lys}B31^{Arg}-insulin;
A0^{Arg}A21^{Ser}B29^{Lys-Nε-Lys}B31^{Arg}-insulin;
A0^{Lys}B29^{Lys-Nε-Arg}-B31^{Lys}insulin;
A0^{Lys}A21^{Xaa}B29^{Lys-Nε-Arg}B31^{Lys}-insulin;
A0^{Lys}A21^{Gly}B29^{Lys-Nε-Arg}B31^{Lys}-insulin; and
A0^{Lys}A21^{Ser}B29^{Lys-Nε-Arg}B31^{Lys}-insulin.

In another preferred embodiment, the insulin molecule of the present invention comprises a modification of the N-terminus of the A-chain and the N-terminus of the B-chain. For example, such an insulin molecule is one in which an Arg has been covalently attached to the N-terminus of the insulin A-chain, and an Arg has been covalently attached to the insulin B-chain. In one preferred embodiment, the present invention provides an insulin molecule having

(a) an A-chain of Formula I,

A-1 A0 A1 A2 A3 A4 A5 A6 A7 A8 A9 A10
Xaa-Xaa-Gly-Ile-Val-Glu-Gln-Cys-Cys-Thr-Ser-Ile-
A11 A12 A13 A14 A15 A16 A17 A18 A19 A20 A21
Cys-Ser-Leu-Tyr-Gln-Leu-Glu-Asn-Tyr-Cys-Xaa,

wherein the amino acid sequence of Formula I is set forth in Seq. ID No. 1, and

(b) a B-chain of Formula II,

B-1 B0 B1 B2 B3 B4 B5 B6 B7 B8 B9 B10
Xaa-Xaa-Phe-Val-Asn-Gln-His-Leu-Cys-Gly-Ser-His-
B11 B12 B13 B14 B15 B16 B17 B18 B19 B20 B21 B22
Leu-Val-Glu-Ala-Leu-Tyr-Leu-Val-Cys-Gly-Glu-Arg-
B23 B24 B25 B26 B27 B28 B29 B30
Gly-Phe-Phe-Tyr-Thr-Xaa-Xaa-Xaa,

wherein the amino acid sequence of Formula II is set forth in Seq. ID No. 2, wherein
Xaa at position A-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position A0 is Arg, derivatized Arg, homoarginine, desamino homoarginine, desaminoarginine, Lys, derivatized Lys, desaminolysine, alpha guanidino homoarginine, or alpha methyl arginine;
Xaa at position A21 is a genetically encodable amino acid;
Xaa at position B-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position B0 is Arg, derivatized Arg, homoarginine, desamino homoarginine, desaminoarginine, Lys, derivatized Lys, desaminolysine, alpha guanidino homoarginine, or alpha methyl arginine;
Xaa at position B28 is Lys or Pro;
Xaa at position B29 is Lys or Pro;
Xaa at position B30 is Thr, Ala or is absent;
one of Xaa at position B28 or Xaa at position B29 is Lys; and
Xaa at position B28 and Xaa at position B29 are not both Lys.

Also provided is a microcrystal comprising this insulin analog and zinc. In a preferred embodiment, the microcrystal comprises the insulin analog, zinc and protamine.

Also provided is a process for preparing the microcrystal, comprising contacting ingredients comprising the insulin molecule and a divalent metal cation in aqueous solvent at a pH that permits formation of hexamers of the insulin molecule. “Contacting” refers broadly to placing the ingredients in solution. Less broadly, contacting refers to the turning, swirling, shaking or vibrating of a solution of the ingredients. More specifically, contacting refers to the mixing of the ingredients.

In another preferred embodiment, the insulin analog is selected from the group consisting of:
A0^{Arg}B0^{Arg}-insulin;
A0^{Arg}A21^{Xaa}B0^{Arg}-insulin;
A0^{Arg}A21^{Gly}B0^{Arg}-insulin;
A0^{Arg}A21^{Ser}B0^{Arg}-insulin;
A0^{Lys}B0^{Lys}-insulin;
A0^{Lys}A21^{Xaa}B0^{Lys}-insulin;
A0^{Lys}A21^{Gly}B0^{Lys}-insulin;
A0^{Lys}A21^{Ser}B0^{Lys}-insulin;
A0^{Arg}B0^{Lys}-insulin;
A0^{Arg}A21^{Xaa}B0^{Lys}-insulin;
A0^{Arg}A21^{Gly}B0^{Lys}-insulin;
A0^{Arg}A21^{Ser}B0^{Lys}-insulin;
A0^{Lys}B0^{Arg}-insulin;
A0^{Lys}A21^{Xaa}B0^{Arg}-insulin;
A0^{Lys}A21^{Gly}B0^{Arg}-insulin; and
A0^{Lys}A21^{Ser}B0^{Arg}-insulin.

“Insulin template” means the insulin molecule that is modified to form an insulin analog or derivative of the present invention. Insulin molecules that can be used as templates for subsequent chemical modification, include, but are not limited to, any one of the naturally occurring insulins, and preferably human insulin; an analog of human insulin; B28^{Lys}, B29^{Pro}-insulin; A0^{Arg}-insulin; A21^{Xaa}-insulin; A0^{Arg}A21a^{Xaa}-insulin, B0^{Arg}-insulin; B28^{ASP}-insulin; B3^{Lys}B29^{Glu}-insulin and an insulin molecule in which one or two free amino groups have been previously derivatized with a protecting group preferably tert-butyloxycarbonyl (Boc) in order to increase the reaction specificity of the subsequent acylation step. Preferably, the insulin template is a recombinant insulin. More preferably, the insulin template is recombinant human insulin or an analog thereof. Most preferably the insulin template is recombinant human insulin.

A21^{Xaa}-insulin can be used as the insulin template if it is desired to replace the wild-type Asparagine at position 21 of Formula I (corresponding to position 23 of Seq. ID No. 2) with another amino acid, in order to diminish or prevent deamidation of the insulin molecule, and/or to prolong the insulin effect of the molecule. In one preferred embodiment, A21^{Asn} is replaced with A21^{Gly} to form A21^{Gly}-insulin. In another preferred embodiment, A21^{Asn} is replaced with A21^{Thr} to form A21^{Thr}-insulin. In another preferred embodiment, A21^{Asn} is replaced with A21^{Ala} to form A21^{Ala}-insulin. In another preferred embodiment, A21^{Asn} is replaced with A21^{Ser} to form A21^{Ser}-insulin.

“Recombinant protein” means a protein that is expressed in a eukaryotic or prokaryotic cell from an expression vector containing a polynucleotide sequence that encodes the protein. Preferably, the recombinant protein is a recombinant insulin molecule.

“Recombinant insulin molecule” is an insulin molecule that is expressed in a eukaryotic or prokaryotic cell from an expression vector that contains polynucleotide sequences that encode the A-chain and B-chain of an insulin molecule, and optionally the C-peptide of a proinsulin molecule. In one preferred embodiment, the recombinant protein is a recombinant insulin or proinsulin derivative. In another preferred embodiment, the recombinant protein is a recombinant insulin or proinsulin analog.

“Recombinant human insulin” means recombinant insulin having the wild-type human A-chain (Seq. ID No. 3) and B-chain (Seq. ID No. 4) amino acid sequences.

“Genetically encodable amino acid” means an amino acid that is encoded by a genetic codon, which is a group of three bases of deoxyribonucleic acid. See Biochemistry, L. Stryer, Ed., Third Edition, W.H. Freeman and Co., New York, p. 99-107 (1988). Genetically encodable amino acids include alanine (Ala), arginine (Arg), asparagine (Asn), aspartic acid (Asp), cysteine (Cys), glutamatic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val).

A clinically normal fasting plasma glucose level is 70-110 mg/dl. A clinically normal postprandial plasma glucose level is less than 140 mg/dl. “Sufficient to regulate blood glucose in a subject” means that administration of an insulin molecule results in a clinically normal fasting plasma glucose level.

As is well-known to those of ordinary skill in the art, insulin effect can be quantified using the “glucose clamp” technique, in which the amount of exogenous glucose required over time to maintain a predetermined plasma glucose level is used as a measure of the magnitude and duration of an insulin effect caused by an insulin molecule. For example, see Burke et al., Diabetes Research, 4:163-167 (1987). Typically, in a glucose clamp investigation, glucose is infused intravenously. If an insulin molecule causes a decrease in plasma glucose level, the glucose infusion rate is increased, such that the predetermined plasma glucose level is maintained. When the insulin molecule effect diminishes, the glucose infusion rate is decreased, such that the predetermined plasma glucose level is maintained.

“Insulin effect” means that in a glucose clamp investigation, administration of an insulin molecule requires that the rate of intravenous blood glucose administration be increased in order to maintain a predetermined plasma glucose level in the subject for the duration of the glucose clamp experiment. In one preferred embodiment, the predetermined glucose level is a fasting plasma glucose level. In another preferred embodiment, the predetermined glucose level is a postprandial plasma glucose level.

An insulin molecule has a “protracted duration of action” if the insulin molecule provides an insulin effect in hyperglycemic, e.g., diabetic, patients that lasts longer than human insulin. Preferably the insulin molecule provides an insulin effect for from about 8 hours to about 24 hours after a single administration of the insulin molecule. More preferably the insulin effect lasts from about 10 hours to about 24 hours. Even more preferably, the effect lasts from about 12 hours to about 24 hours. Still more preferably, the effect lasts from about 16 hours to about 24 hours. Most preferably, the effect lasts from about 20 hours to about 24 hours.

An insulin molecule has a “basal insulin effect” if the insulin molecule provides a glucose lowering effect in subjects that lasts about 24 hours after a single administration of the insulin molecule.

“Isolated protein” as used herein means that the protein is removed from the environment in which it is made. A naturally occurring protein is isolated when it is removed from the cellular milieu in which the protein exists. A recombinant protein is isolated when it is removed from the cellular milieu in which the protein is expressed. A chemically modified protein, whether naturally occurring or recombinant, is isolated when it is removed from the reaction mixture in which the protein is chemically modified. Preferably, an isolated protein is removed from other proteins, polypeptides, or peptides. Methods for isolating a protein include centrifugation, chromatography, lyophilization, or electrophoresis. Such protein isolation methods and others are well known to those of ordinary skill in the art. Preferably, the insulin molecule of the present invention is isolated.

“Modification” of a protein refers to the addition of an amino acid or derivatized amino acid, to the substitution of one amino acid by another, or to the deletion of an amino acid. Modification can be accomplished via recombinant DNA methodology. For example, see U.S. Pat. Nos. 5,506,202, 5,430,016, and 5,656,782. Alternatively, modification can be accomplished via chemical modification of an insulin template, such as by adding one or more chemical moieties to an insulin template, or removing one or more chemical moieties from an insulin template. Chemical modifications at insulin template amino acid side groups include carbamylation, amidation, guanidinylation, sulfonylation, acylation of one or more α-amino groups, acylation of an ε-amino group (e.g., a lysine ε-amino group), N-alkylation of arginine, histidine, or lysine, alkylation of glutamic or aspartic carboxylic acid groups, and deamidation of glutamine or asparagine. Modifications of a terminal amino group (e.g., an α-amino group) include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist.

Amino acids used to make the insulin analog or insulin derivative of the present invention can be either the D- or L-form, and can be either naturally-occurring amino acids or artificial amino acids.

“Derivatized Arg” means an Arginine that has been modified via a synthetic chemical approach. Preferred Arg derivatives are obtained through acylation and/or carbamylation. In a preferred embodiment, Arg is derivatized with a positively charged amino acid. In another preferred embodiment, Arg is derivatized with Arg at the epsilon (—Nε) amino group to form Arg-Nε-Arg. In another preferred embodiment, Arg is derivatized with Lys at the —Nε amino group to form Arg-Nε-Lys. In another preferred embodiment, the derivatized Arg is dArgine (dArg or dR), which is Arg with inverted stereochemistry at the alpha carbon.

“Derivatized Lys” means a Lysine that has been modified via a synthetic chemical approach. Preferred Lys derivatives are obtained through acylation and/or carbamylation. In a preferred embodiment, Lys is derivatized with a positively charged amino acid. In another preferred embodiment, Lys is derivatized with Arg at the epsilon (—Nε) amino group to form Lys-Nε-Arg. In another preferred embodiment, Lys is derivatized with Lys at the epsilon amino group to form Lys-Nε-Lys. In another preferred embodiment, the derivatized Lys is homoarginine (homoArg or hR). In another preferred embodiment, the derivatized Lys is dLysine (dLys or dL), which is Lys with inverted stereochemistry at the alpha carbon. In another preferred embodiment, the derivatized Lys is alpha guanidino homoarginine (gHR).

Human insulin contains three free amino groups: the N-terminal α-amino group of the A-chain, the N-terminal α-amino group of the B-chain, and the ε-amino group of a B-chain lysine side chain. Generally, the α- and/or ε-amino groups of proteins can be acylated with activated carboxylic acids. In this context, acylation refers to the formation of an amide bond between the amine and the carboxylic acid.

Acylation of the N-termninal amino acid of the insulin A-chain with an amino acid results in the formation of a peptide bond. Likewise, acylation of the N-terminal amino acid of the insulin B-chain with an amino acid results in the formation of a peptide bond. Acylation of the ε-amino group of a Lys with an amino acid forms the Lys-Nε-amino acid derivative.

“Acylated Arg” refers to an acyl moiety that is covalently bound to Arg through a covalent bond formed between the acid group of an acyl-containing compound and the ε-amino group of Arg.

“Acylated Lys” refers to an acyl moiety that is covalently bound to Lys through a covalent bond formed between the acid group of an acyl-containing compound and Lys.

“Carbamylated insulin” means a carbamyl moiety that is covalently bound to insulin through a covalent bond formed between the carbonyl carbon of the carbamyl group of a carbamyl-containing compound and an amino group of insulin.

“Carbamylated Arg” refers to a carbamyl moiety that is covalently bound to Arg through a covalent bond formed between the carbonyl carbon of the carbamyl group of a carbamyl-containing compound and the alpha-amino group of Arg.

“Carbamylated Lys” refers to a carbamyl moiety that is covalently bound to Lys through a covalent bond formed between the carbonyl carbon of the carbamyl group of a carbamyl-containing compound and Lys.

“Pharmaceutically acceptable” means clinically suitable for administration to a human. A pharmaceutically acceptable formulation does not contain toxic elements, undesirable contaminants or the like, and does not interfere with the activity of the active compounds therein.

“Pharmaceutical composition” means a composition that is clinically acceptable to administer to a human subject. The insulin molecule of the present invention can be formulated in a pharmaceutical composition such that the protein interacts with one or more inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

“Microcrystal” means a solid that comprises primarily matter in a crystalline state, and of a microscopic size, typically of longest dimension within the range 1 micron to 100 microns. “Microcrystalline” refers to the state of being a microcrystal.

“Amorphous precipitate” refers to insoluble material that is not crystalline in form. The person of ordinary skill can distinguish crystals from amorphous precipitate.

“Suspension” means a mixture of a liquid phase and a solid phase that consists of insoluble or sparingly soluble particles that are larger than colloidal size. For example, mixtures of NPH microcrystals and an aqueous solvent form suspensions.

“Suspension formulation” means a pharmaceutical composition wherein an active agent is present in a solid phase, for example, a microcrystalline solid, an amorphous precipitate, or both, which is finely dispersed in an aqueous solvent. The finely dispersed solid is such that it may be suspended in a fairly uniform manner throughout the aqueous solvent by the action of gently agitating the mixture, thus providing a reasonably uniform suspension from which a dosage volume may be extracted. Examples of commercially available insulin suspension formulations include, for example, NPH, PZI, and Ultralente. A small proportion of the solid matter in a microcrystalline suspension formulation may be amorphous. Preferably, the proportion of amorphous material is less than 10%, and most preferably, less than 1% of the solid matter in a microcrysialline suspension. Likewise, a small proportion of the solid matter in an amorphous precipitate suspension may be microcrystalline.

“Protamine” means a mixture of strongly basic proteins obtained from fish sperm. The average molecular weight of the proteins in protamine is about 4,200 (Hoffmann, J. A., et al., Protein Expression and Purification, 1:127-133 (1990)]. “Protamine” can refer to a relatively salt-free preparation of the proteins, often called “protamine base.’ Protamine also refers to preparations comprised of salts of the proteins, e.g., protamine sulfate. Commercial preparations vary widely in their salt content.

“Aqueous solvent” means a liquid solvent that contains water. An aqueous solvent system may be comprised solely of water, may be comprised of water plus one or more miscible solvents, or may contain solutes. Commonly-used miscible solvents are the short-chain organic alcohols, such as methanol, ethanol, propanol; short-chain ketones, such as acetone; and polyalcohols, such as glycerol.

“Isotonicity agent” means a compound that is physiologically tolerated and imparts a suitable tonicity to a formulation to prevent the net flow of water across cell membranes that are in contact with an administered formulation. Glycerol, which is also known as glycerin, and mannitol, are commonly used isotonicity agents. Other isotonicity agents include salts, e.g., sodium chloride, and monosaccharides, e.g., dextrose and lactose. Preferably the isotonicity agent is glycerol.

“Hexamer-stabilizing compound” means a non-proteinaceous, small molecular weight compound that stabilizes the insulin molecule of the present invention in a hexameric association state. Phenolic compounds, particularly phenolic preservatives, are the best known stabilizing compounds for insulin molecules. Preferably, the hexamer-stabilizing compound is one of phenol, m-cresol, o-cresol, p-cresol, chlorocresol, methylparaben, or a mixture of two or more of those compounds. More preferably, the hexamer-stabilizing compound is phenol or m-cresol, or a mixture thereof.

“Preservative” refers to a compound added to a pharmaceutical formulation to act as an anti-microbial agent. Tile preservative used in formulations of the present invention may be a phenolic preservative, and may be the same as, or different from the hexamer-stabilizing compound. A parenteral formulation must meet guidelines for preservative effectiveness to be a commercially viable multi-use product. Among preservatives known in the art as being effective and acceptable in parenteral formulations are benzalkonium chloride, benzethonium, chlorohexidine, phenol, m-cresol, benzyl alcohol, methylparaben, chlorobutanol, o-cresol, p-cresol, chlorocresol, phenylmercuric nitrate, thimerosal, benzoic acid, butyl paraben, ethyl paraben, phenoxy ethanol, a phenyl ethylalcohol, propyl paraben, benzylchlorocresol, chlorocresol, and various mixtures thereof.

“Phenolic preservative” includes the compounds phenol, m-cresol, o-cresol, p-cresol, chlorocresol, methylparaben, and mixtures thereof. Certain phenolic preservatives, such as phenol and m-cresol, are known to bind to insulin-like molecules and thereby to induce conformational changes that increase either physical or chemical stability, or both. Preferably, the phenolic preservative is m-cresol or phenol. “Buffer” or “pharmaceutically acceptable buffer” refers to a compound that is safe for use in insulin formulations and that has the effect of controlling the pH of the formulation at the pH desired for the formulation. The pH of the crystalline formulation of the present invention is from about 6.0 to about 8.0. The pH of the solution formulation of the present invention is from about 3.5 to about 6.0.

Pharmaceutically acceptable buffers for controlling pH at a moderately acidic pH to a moderately basic pH include such compounds as lactate; tartrate; phosphate, and particularly sodium phosphate; acetate, and particularly sodium acetate; citrate, and particularly sodium citrate; arginine; TRIS; and histidine. “TRIS” refers to 2-amino-2-hydroxymethyl-1,3,-propanediol, and to any pharmacologically acceptable salt thereof. The free base and the hydrochloride form are two common forms of TRIS. TRIS is also known in the art as trimethylol aminomethane, tromethamine, and tris(hydroxymethyl)aminomethane. Other pharmaceutically acceptable buffers that are suitable for controlling pH at the desired level are known to the chemist of ordinary skill.

A “rapid-acting insulin analog” provides a hypoglycemic effect that (a) begins sooner after subcutaneous administration than human insulin, and/or (b) exhibits a shorter duration of action than human insulin after subcutaneous administration. B28^{Lys}B29^{Pro}-insulin (so-called “lispro” insulin) is a rapid-acting insulin analog, in which the Pro at position 28 of the wild-type insulin B-chain (Seq. ID No. 4) and the Lys at position 29 of the wild-type insulin B-chain (Seq. ID No. 4) have been switched. See, for example, U.S. Pat. Nos. 5,504,188 and 5,700,662. Another rapid-acting insulin analog is B28^{Asp}-insulin, in which the wild-type Pro at position 28 of the B-chain has been replaced by Asp. See U.S. Pat. No. 6,221,633. Another rapid-acting insulin analog is B3^{Lys}B29^{Glu}-insulin. See U.S. Pat. No. 6,221,633.

Also provided herein is a microcrystal comprising an insulin molecule of the present invention. In one embodiment, the microcrystal does not contain protamine. In another aspect of the invention, the microcrystal does not contain protamine and does contain a divalent cation, e.g., zinc. Such a crystal is particularly suited for making bulk crystals in solution or in dried form for subsequent formulation.

In another embodiment, the microcrystal contains protamine.

In another embodiment, the microcrystal contains both an insulin molecule of the present invention and human insulin. In one preferred embodiment, the microcrystal is used to make a solution formulation. In another preferred embodiment, the microcrystal is used to make a suspension formulation

Also provided is a suspension formulation comprising an insulin molecule of the present invention. Also provided is a composition comprising the suspension formulation. In one embodiment, the suspension formulation contains an insoluble phase and a solution phase, the insoluble phase comprising the microcrystal of the invention, and the solution phase comprising water. If desired, the solution phase contains human insulin or a rapid-acting insulin analog, such as B28^{Lys}B29^{Pro}-insulin, B28^{Asp}-insulin, or B3^{Lys}B29^{Glu}.

The suspension formulation can be used to prepare a medicament for the treatment of diabetes mellitus. The suspension formulation can also be used to treat diabetes mellitus, in a method comprising administering the suspension formulation to a subject in an amount sufficient to regulate blood glucose concentration in the subject.

The insulin molecule of the present invention can be complexed with a suitable divalent metal cation. “Divalent metal cation” means the ion or ions that participate to form a complex with a multiplicity of protein molecules. The transition metals, the alkaline metals, and the alkaline earth metals are examples of metals that are known to form complexes with insulin. The transitional metals are preferred. Preferably, the divalent metal cation is one or more of the cations selected from the group consisting of zinc, copper, cobalt, manganese, calcium, cadmium, nickel, and iron. More preferably, zinc is the divalent metal cation. Preferably, zinc is provided as a salt, such as zinc sulfate, zinc chloride, zinc oxide, or zinc acetate. Divalent metal complexes of the insulin molecule are generally insoluble in aqueous solution around physiological pH. Thus, these complexes can be administered subcutaneously as suspensions and show a decreased rate of release in vivo, thereby extending the time action of the compound.

To obtain the complexes between the insulin molecule of the present invention and a divalent metal cation, the protein is dissolved in a suitable buffer and in the presence of a metal salt. The mixture is allowed to incubate at ambient temperature to allow the complex to precipitate. Suitable buffers are those which maintain the mixture at a pH range from about 3.0 to about 9.0 and do not interfere with the complexation reaction. Examples include phosphate buffers, acetate buffers, citrate buffers and Goode's buffers, e.g., HEPES, Tris and Tris acetate. Suitable metal salts are those in which the metal is available for complexation. Examples of suitable zinc salts include zinc chloride, zinc acetate, zinc oxide, and zinc sulfate.

“Protected amino acid” is an amino acid having all but one of the reactive functional groups reversibly derivatized, such that only one functional group is reactive. For example, for a protected, activated carboxylic acid, the alpha carboxylate group is reactive, but all other functional groups on the activated carboxylic acid are non-reactive. A protected amino acid is “deprotected” when the protecting functionality is removed. Preferably, the protected amino acid is protected arginine.

A “conservative substitution” is the replacement of an amino acid with another amino acid that has the same net electronic charge and approximately the same size and shape. Amino acids with aliphatic or substituted aliphatic amino acid side chains have approximately the same size when the total number carbon and heteroatoms in their side chains differs by no more than about four. They have approximately the same shape when the number of branches in the their side chains differs by no more than one. Amino acids with phenyl or substituted phenyl groups in their side chains are considered to have about the same size and shape. Listed below are five groups of amino acids. Replacing an amino acid in insulin with another amino acid from the same groups results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutaimic acid, aspartic acid and non-naturally occurring amino acids with carboxylic acid substituted C1-C4 aliphatic side chains (unbranched or one branch point).
Group III: lysine, ornithine, arginine, homoarginine, and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

Except as otherwise specifically provided herein, conservative substitutions are preferably made with naturally occurring amino acids.

A “highly conservative substitution” is the replacement of an amino acid with another amino acid that has the same functional group in the side chain and nearly the same size and shape. Amino acids with aliphatic or substituted aliphatic amino acid side chains have nearly the same size when the total number carbon and heteroatoms in their side chains differs by no more than two. They have nearly the same shape when they have the same number of branches in the their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

In an insulin molecule of the present invention, the A-chain can have an additional 1-3 amino acids at the A-chain C-terminus, which would be positions A22, A23 and A24 of formula I. Preferably, the amino acid at each of positions A22, A23 and A24 are Xaa, wherein Xaa is a genetically encodable amino acid.

The B-chain can have an additional 1-6 amino acids at the B-chain C-terminus, which would be positions B31, B32, B33, B34, B35 and B36 of formula II. In one preferred embodiment, the B-chain comprises Ala at position B31, Arg at position B32, and Arg at positions B33. In another preferred embodiment, the B-chain comprises Ala at position B31, Ala at position B32, Ala at position B33, Ala at position B34, Arg at position B35, and Arg at position B35.

An “effective amount” of the insulin molecule, microcrystal, suspension, solution amorphous precipitate or compositions of the present invention is the quantity which results in a desired insulin effect without causing unacceptable side-effects when administered to a subject in need of insulin therapy. An “effective amount” of the insulin molecule of the present invention administered to a subject will also depend on the type and severity of the disease and on the characteristics of the subject, such as general health, age, sex, body weight and tolerance to drugs. The skilled artisan will be able to determine appropriate dosages depending on these and other factors. Typically, a therapeutically effective amount of the insulin molecule of the present invention can range from about 0.01 mg per day to about 1000 mg per day for an adult. Preferably, the dosage ranges from about 0.1 mg per day to about 100 mg per day, more preferably from about 1.0 mg/day to about 10 mg/day.

A “desired therapeutic effect” includes one or more of the following: 1) an amelioration of the symptom(s) associated with diabetes mellitus, 2) a delay in the onset of symptoms associated with diabetes mellitus, 3) increased longevity compared with the absence of the treatment, and 4) greater quality of life compared with the absence of the treatment. For example, an “effective amount” of the insulin molecule of the present invention for the treatment of diabetes is the quantity that would result in greater control of blood glucose concentration than in the absence of treatment, thereby resulting in a delay in the onset of diabetic complications such as retinopathy, neuropathy or kidney disease.

The dose, route of administration, and the number of administrations per day will be determined by a physician considering such factors as the therapeutic objectives, the nature and cause of the patient's disease, the patient's gender and weight, level of exercise, eating habits, the method of administration, and other factors known to the skilled physician. In broad range, a daily dose would be in the range of from about 1 nmol/kg body weight to about 6 nmol/kg body weight (6 nmol is considered equivalent to about 1 unit of insulin activity). A dose of between about 2 and about 3 nmol/kg is typical of present insulin therapy.

The physician of ordinary skill in treating diabetes will be able to select the therapeutically most advantageous means to administer the formulations of the present invention. Parenteral routes of administration are preferred. Typical routes of parenteral administration of solution and suspension formulations of insulin are the subcutaneous and intramuscular routes. The compositions and formulations of the present invention may also be administered by nasal, buccal, pulmonary, or occular routes.

The insulin molecule of the present invention, and compositions thereof, can be administered parenterally. Parenteral administration can include, for example, systemic administration, such as by intramuscular, intravenous, subcutaneous, or intraperitoneal injection. Preferably, the route of administration is subcutaneous.

The insulin molecule of the present invention, and compositions thereof, can be administered to the subject in conjunction with one or more pharmaceutically acceptable excipients, carriers or diluents as part of a pharmaceutical composition for treating hyperglycemia.

The insulin molecule of the present invention, and compositions thereof, can be a solution. Alternatively, the insulin molecule of the present invention, and compositions thereof, can be a suspension of the insulin molecule of the present invention or a suspension of the protein compound complexed with a divalent metal cation.

Also provided herein is a composition comprising an insulin molecule of the present invention and at least one ingredient selected from the group consisting of an isotonicity agent, a divalent cation, a hexamer-stabilizing compound, a preservative, and a buffer.

Suitable pharmaceutical carriers may contain inert ingredients which do not interact with the insulin molecule of the present invention. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include glycerol, lactose, dextrose, sucrose, trehalose, sorbitol, and mannitol.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

An insulin template and an insulin analog can be obtained using recombinant methodologies. For example, a recombinant proinsulin or proinsulin analog can be used. Alternatively, recombinant insulin A- and B-chains can be expressed in host cells and then recombined. Alternatively, an insulin precursor can be used. Each of these methodologies are well known to those of ordinary skill in the art. For example, see U.S. Pat. No. 4,421,685, U.S. Pat. No. 4,569,791, U.S. Pat. No. 4,569,792, U.S. Pat. No. 4,581,165, U.S. Pat. No. 4,654,324, U.S. Pat. No. 5,304,473, U.S. Pat. No. 5,457,066, U.S. Pat. No. 5,559,094 European patent application EP 741188 A1. See also Chance et al., Diabetes Care 16 (Suppl 3): 133-142 (1993); Chance et al., “Peptides: Synthesis-Structure-Function,” in: Proceedings of the 7^{th} American Peptide Symposium, Rich, D. H. et al., eds., Pierce Chemical Company, Rockford, Ill., pp. 721-738 (1981); and Frank et al., Munch med Wsch 125 (Suppl. 1): S14-20 (1983).

In one preferred embodiment, A0^{Lys-Nε-Arg}A21^{Gly}B29^{Lys-Nε-Arg}-insulin is made by selectively acylating the ε-amino groups of A0^{Lys}A21^{Gly}B29^{Lys}-insulin. Selective acylation of ε-amino groups can be accomplished by those of ordinary skill in the art. For example, see U.S. Pat. No. 5,646,242. In another preferred embodiment, A0^{Lys-Nε-Arg}A21^{Gly}B29^{Lys-Nε-Arg}-insulin is made by selectively acylating the ε-amino groups of A21^{Gly}C64^{Arg}C65^{Lys}-human proinsulin and digesting the acylated proinsulin derivative with proteases to remove undesired amino acids, while keeping intact the C65^{Lys-Nε-Arg} and B29^{Lys-Nε-Arg} to form the A0^{Lys-Nε-Arg}A21^{Gly}B29^{Lys-Nε-Arg}-insulin derivative.

Recombinant insulin molecules can be produced by a method which comprises culturing a host cell containing a DNA sequence encoding the insulin molecule or a precursor thereof and capable of expressing the polypeptide in a suitable nutrient medium under conditions permitting the expression of the peptide, after which the resulting peptide is recovered from the host cells and/or from the culture medium.

The medium used to culture the cells may be any conventional medium suitable for growing the host cells, such as minimal or complex media containing appropriate supplements. Suitable media are available from commercial suppliers or may be prepared of published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of peptide in question.

Accordingly, provided herein is a method of expressing an insulin molecule of the present invention, comprising cultivating a host cell containing the insulin molecule under conditions suitable for propagation of the host cell and for expression of the insulin molecule. In one preferred embodiment, the method further comprises purifying the insulin molecule from the host cell. In another preferred embodiment, the method further comprises purifying the insulin molecule from the culture medium. In yet another preferred embodiment, the method further comprises purifying the insulin molecule from both the host cell and from the culture medium.

In a preferred embodiment, the host cell is a eukaryotic cell. Preferably, the eukaryotic cell is a fungal cell, a yeast cell, a mammalian cell, or an immortalized mammalian cell line cell. In another preferred embodiment, the host cell is a prokaryotic cell. Preferably, the eukaryotic cell is a bacterial cell, and more preferably is an E. coli cell.

Nucleic acid sequence encoding the insulin molecule or precursor thereof may be inserted into any vector which may conveniently be subjected to recombinant DNA procedures, and the choice of vector will often depend on the host cell into which it is to be introduced. Thus, the vector may be an autonomously replicating vector, i.e., a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the peptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art.

The DNA sequence encoding the peptide may also, if necessary, be operably connected to a suitable terminator, polyadenylation signals, transcriptional enhancer sequences, and translational enchancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracyclin, chloramphenicol, neomycin, hygromycin or methotrexate.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The insulin molecule of the present invention can be prepared by using standard methods of solid-phase peptide synthesis techniques. Peptide synthesizers are commercially available from, for example, Applied Biosystems in Foster City Calif. Reagents for solid phase synthesis are commercially available, for example, from Midwest Biotech (Fishers, Ind.). Solid phase peptide synthesizers can be used according to manufacturers instructions for blocking interfering groups, protecting the amino acid to be reacted, coupling, decoupling, and capping of unreacted amino acids.

Typically, an α-N-carbamyl protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-carbamyl protecting group is removed from the resulting peptide resin using a reagent such as trifluoroacetic acid (TFA) or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991, the entire teachings of which are incorporated by reference. Examples include t-butyloxycarbonyl (tBoc) and fluorenylmethoxycarbonyl (Fmoc).

Peptides can be synthesized using standard automated solid-phase synthesis protocols using t-butoxycarbonyl- or fluorenylmethoxycarbonyl-alpha-amino acids with appropriate side-chain protection. After completion of synthesis, peptides are cleaved from the solid-phase support with simultaneous side-chain deprotection using standard hydrogen fluoride or TFA methods. Crude peptides are then further purified using Reversed-Phase Chromatography on Vydac C18 columns using linear water-acetonitrile gradients in which all solvents contain 0.1% TFA. To remove acetonitrile and water, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The insulin molecule of the present invention can be made by chemically modifying a recombinant insulin template. In one embodiment, the recombinant insulin template is acylated with one or more protected amino acids using an activated carboxylic acid moiety. Preferably, an activated ester or amide is used. More preferably, an activated ester is used. Even more preferably, an N-hydroxysuccinimide (NHS) ester is used.

In a method of the present invention, an insulin molecule is made by chemically modifying an insulin template, such that the insulin template is acylated with protected amino acids using an activated carboxylic acid moiety. Preferably, an activated ester or amide is used. More preferably, an activated ester is used. Even more preferably, an N-hydroxysuccinimide (NHS) ester is used. Techniques for acylating the N-terminus of an insulin A-chain and/or a B-chain Lys are well known to those of ordinary skill in the art.

Thus, in one preferred embodiment, recombinant A21^{Xaa}-insulin is acylated at the A1 and B29 positions to form A0^{Arg}A21^{Xaa}B29^{Lys-Nε-Arg}-insulin. In another preferred embodiment, A21^{Gly}-insulin is acylated at the A1 and B29 positions to form A0^{Arg}A21^{Gly}B29^{Lys-Nε-Arg}-insulin.

In another preferred embodiment, recombinant A0^{Arg}A21^{Xaa}-insulin is acylated at the B29 position to form A0^{Arg}A21^{Xaa}B29^{Lys-Nε-Lys}-insulin. In another preferred embodiment, A0^{Arg}A21^{Gly}-insulin is acylated at the B29 position to form A0^{Arg}A21^{Gly}B29^{Lys-Nε-Lys}-insulin.

In another preferred embodiment, recombinant A0^{Lys}A21^{Xaa}-insulin is acylated at the A0 and B29 positions to form A0^{Lys-Nε-Arg}A21^{Xaa}B29^{Lys-Nε-Arg}-insulin. Preferably, A0^{Lys}A21^{Gly}-insulin is acylated at the A0 and B29 positions to form A0^{Lys-Nε-Arg}A21^{Gly}B29^{Lys-Nε-Arg}-insulin.

In another preferred embodiment, a proinsulin analog is used to make the insulin molecule of the present invention. In wild-type human proinsulin, a Lys is at amino acid position 64, and an Arg is at amino acid position 65. A proinsulin analog having an Arg at position 64 and a Lys at position 65 can be used to generate A0^{Lys}A21^{Xaa}-insulin, which is then acylated at the A0 and B29 positions to form A0^{Lys-Nε-Arg}A21^{Xaa}B29^{Lys-Nε-Arg}-insulin. Preferably, A0^{Lys}A21^{Gly}-insulin is acylated at the B29 position to form A0^{Lys-Nε-Arg}A21^{Gly}B29^{Lys-Nε-Arg}-insulin.

Protein acylation reactions are preferably carried out in mixtures of water and organic solvents, but can also be done in pure organic or purely aqueous conditions, depending on reactant solubility. In the following examples, reactions were carried out in mixtures containing between 40 and 60% organic with MeOH, DMF or CH₃CN as the organic component. The activated carboxylic acid moieties comprise amino acids, dipeptides or short polypeptides in which the ε-amino group and all side chain functional groups are derivatized with appropriate protecting groups, which preferably are removed after the protein derivatization step is complete. Preferably, the carboxylate activating group is N-hydroxy-succinimide (NHS), due to its favorable solubility in aqueous mixtures and the reactivity of the resulting NHS-esters with protein amino groups. The ratio of the NHS-ester to insulin template can vary between 2 and 20, but preferably is between 3 and 5. The ratio is adjusted based on the desired extent of mono-, di-, and tri-acylated product(s), as well as the relative reactivity of the incoming NHS-ester reagent.

Reactions are carried out at room temperature (20-25 degrees C.), generally with stirring by a magnetic stir bar or mixing on a rotisserie. Reactions are preferably allowed to progress for ½ hr to 6 hr.

The reaction mixtures are quenched after the desired level of acylation has occurred (as determined by LC-MS monitoring) by acidification with acetic acid or trifluoroacetic acid. Further work-up/purification can be carried out by (1) directly purifying reaction mixtures by reversed-phase HPLC, followed by protecting group removal and re-purification of the resulting isolated, deprotected product(s) by reversed-phase HPLC, or (2) diluting the reaction mixture with water to an organic content of under 25% and lyophilization, followed by protecting group removal, purification by cation exchange chromatography, and final purification/de-salting on reversed-phase HPLC or gel filtration.

Protecting groups can include groups for which deprotection can be carried out in conditions that are compatible with proteins and peptides (i.e., conditions which are not so harsh as to destroy the protein/peptide). For example, tert-butyloxycarbonyl (Boc) or trifluoroacetyl (tfa) groups can be used to protect amino functionalities. The protecting groups can be removed, for example, with trifluoroacetic acid (TFA) and aqueous ammonium hydroxide (NH₄OH), respectively. Protection of the guanidino moiety is via Boc, Pmc (2,2,5,7,8-Pentamethylchroman-6-sulfonyl), or Pbf (2,2,4,6,7-Pentamethylbenzofuran-5-sulfonyl) groups. The Pmc and Pbf groups are also removed with TFA but require the presence of scavengers, as described further in the examples below.

Because amino groups must be in the neutral (deprotonated) form to react appreciably in the acylation, the pH at which the reaction is carried out greatly affects the reaction rate. Generally, in aqueous mixtures, the reaction rate of a particular amino group is inversely related to its pKa, except at very high pH. The reaction rate can also be affected by steric and proximity effects of adjacent residues and by the degree of accessibility of the side chain to solvent. In the case of insulin, the three amines have characteristic pKa values and different effects of the surrounding environment on reactivity which allow some specificity to be achieved (see Lindsey et al., in Biochem. J. 121:737-745 (1971)). In particular, the ε-amino group of the B29:Lysine side chain dominates the acylation reaction at pH above 10 (see Baker et al. U.S. Pat. No. 5,646,242). In the following examples, reactions were performed at pH values ranging from approximately 6 to 11 to allow for fine-tuning of the reaction specificity, depending on the particular product which is desired.

In the following examples, the identities of the final products were confirmed by a combination of techniques which include LC-MS (verification of molecular weight), N-terminal protein sequencing, and LC-MS analysis of S. Aureus V8 protease digest, which yields characteristic insulin fragments due to specific cleavage by this enzyme of peptide bonds on the carboxyl side of Glu residues (see Nakagawa, S. H. & Tager, H. S. in J. Biol. Chem. 266:11502-11509 (1991)).

### EXAMPLE 1

### Acylation of B28^{Lys}B29^{Pro}-Insulin W with Boc-Arg(Boc)₂-NHS Ester in Water/CH₃CN to Produce A0^{Arg}B0^{Arg}B28^{Lys-Nε-Arg}B29^{Pro} Insulin

B28^{Lys}B29^{Pro}-insulin-Zn crystals (320 mg; 0.055 mmol) were dissolved in 30 mL of 1:1 CH₃CN:PBS-buffer. 5M KOH solution was added (50 μL) to dissolve the crystals at pH 10. The pH was then adjusted to approximately 7.5 with 5 M phosphoric acid. Boc-Arg(Boc)₂-NHS ester was prepared from 1 mmol each of Boc-Arg(Boc)₂-OH, NHS, and dicyclohexylcarbo-diimide (DCC) mixed together in dichloromethane for 30 min. The mixture was then filtered and concentrated to dryness on a rotary evaporator. The Boc-Arg(Boc)₂-NHS ester was then dissolved in 4 mL MeOH. 2 mL of Boc-Arg(Boc)₂-NHS ester solution was added to the insulin solution and the solution was mixed at room temperature for 2 hr. The pH at this point had dropped to approximately 6.4. Addition of 40 μL of 5M KOH solution increased the pH back to 7.1. The remaining Boc-Arg(Boc)₂-NHS ester was added to the insulin mixture and the reaction was continued for an additional 2.5 hr. The mixture was then acidified with 100 μL trifluoroacetic acid (TFA), diluted with 30 mL of water, and lyophilized overnight. To generate the deprotected product, the lyophilized material totaling approximately 900 mg, due to the presence of excess acylating reagent and salts from the PBS buffer, was dissolved in 20 mL of TFA and allowed to sit at room temperature for 1 hr. The mixture was then evaporated to near dryness on a rotary evaporator and redissolved in 20 mL of 1:9 CH₃CN:water.

The sample was analyzed by analytical reversed-phase HPLC on a Zorbax Eclipse XDB-C8 4.6 mm i.d.×15 cm column with a linear AB gradient of 10 to 100% B over 15 min in which A=0.05% TFA/H₂O and B=0.05% TFA in 60:40 CH₃CN:H₂O and the flow rate was 1 mL/min. Under these conditions, the sample displayed a major peak confirmed by LC-MS to be the MW of the tri-acylated insulin, with smaller amounts of tetra-acylated and di-acylated insulin eluting just before and just after the main peak, respectively. The relative amounts of the products were not determined since they were not fully resolved under these chromatographic conditions, but approximately 70% of the material appears to be the tri-acylated species.

Half the crude acylated material was purified by cation exchange chromatography on a glass 2 cm i.d.×30 cm column packed with SP-Sepharose material. A linear AB gradient of 0to 40% B was carried out over 100 min with a flow rate of 3 mL/min. The solvent components were A: 70 mM sodium acetate in H₂O:CH₃CN 70:30, pH 4.0 and B: 70 mM sodium acetate, 1 M sodium chloride in H₂O:CH₃CN 70:30, pH 4.0. The fractions containing the tri-acylated insulin product were pooled and the solution was concentrated from approx. 96 mL to 75 mL, diluted back to 100 mL with H₂O and loaded on a Vydac C₁₈ 2.0 cm i.d.×25 cm preparative column at 20 mL/min. The sample was eluted with a flow rate of 10 mL/min using a two-stage linear AB gradient of 0 to 15% B over 15 min followed by 15 to 65% B over 100 min, where A=0.05% TFA/H₂O and B=0.05% TFA/CH₃CN. The combined purified material was lyophilized to give 52 mg, corresponding to an overall yield of approximately 34%.

### EXAMPLE 2

### Acylation of A0^{Arg}-Insulin with Boc-Arg(Boc)₂-NHS Ester in Water/CH₃CN to Produce A0^{Arg}B29^{Lys-Nε-Arg}-Insulin

Boc-Arg(Boc)₂-NHS ester (0.5 mmol) was prepared and dissolved in 5 mL of MeOH. 104 mg of A0^{Arg}-insulin (0.017 mmol) was dissolved in 10 mL of 1:1 PBS buffer/CH₃CN, adjusted to pH 11 with 5 M KOH solution. 0.52 mL of the Boc-Arg(Boc)₂-NHS ester solution (0.052 mmol) was added to the insulin solution. The pH dropped to approximately 9 and was immediately adjusted back to 11 with 5 M KOH solution.

The reaction was allowed to proceed for 30 min at room temperature followed by acidification with 200 μL acetic acid. One major peak was present on analytical HPLC (performed as in EXAMPLE 1 above) with the correct MW for mono-acylated product as determined by LC-MS. The sample was purified directly by reversed-phase HPLC on a Vydac C₁₈ prep. column as described above with a two-stage linear AB gradient of 0 to 18% B over 15 min followed by 18 to 100% B over 160 min. The pooled fractions containing the product were lyophilized and totaled about 61 mg. The lyophilized sample was dissolved in 10 mL TFA and allowed to sit for 30 min, then concentrated to near dryness and redissolved in 20 mL of 10:90 CH₃CN:H₂O. The sample was then submitted to a final reversed-phase purification as described in Example 1 above. The final lyophilized mass was 31 mg for an overall yield of approximately 30%.

### EXAMPLE 3

### Acylation of Recombinant Human Insulin with Boc-Arg(Pbf)Arg(Pbf)-NHS Ester in Water/DMF to produce A-1^{Arg}A0^{Arg}-Insulin

Boc-Arg(Pbf)Arg(Pbf)-NHS ester (0.2 mmol) was prepared from 0.2 mmol each of Boc-Arg(Pbf)Arg(Pbf)-OH, NHS, and dicyclohexylcarbodiimide (DCC) mixed in dichloromethane for 60 min. The sample was then filtered, evaporated to dryness and redissolved in 4 mL DMF. Recombinant human insulin-Zn crystals (320 mg; 0.055 mmol) were dissolved in 20 mL of 1:1 DMF:PBS-buffer. 5M KOH solution was added (50 μL to dissolve the crystals at pH 10. The pH was adjusted to 8.2 with 5 M phosphoric acid, and 3 mL of the Boc-Arg(Pbf)Arg(Pbf)-NHS ester solution (0.15 mmol) was added. After mixing for approximately 1 hr, analytical HPLC (performed as in Example 1 above) showed two peaks due to monoacylated products which were confirmed by LC-MS. The peaks were present in an approximately 70:30 ratio.

Subsequent LC-MS analysis of S. ureus V8 protease digests proved that the more abundant peak was due to acylation of the A-chain N terminus and the smaller peak contained mixture of species which were acylated at either the B-chain N terminus or the side chain amine of B29:Lys. Purification on a Vydac C₁₈ column as in Example 1 yielded 49 mg of the protected A-chain acylated product. This material was deprotected with a mixture of 10 mL of 94:2:2:2 TFA:anisole:MeOH:triisopropylsilane (TIPS) for 1 hr at room temperature.

The mixture was then concentrated to near dryness and redissolved in 6 mL of 20:80 CH₃CN:H₂O, which was extracted twice with 10 mL diethyl ether. Final reversed-phase HPLC purification (as in Example 1) yielded 34 mg of A-1^{Arg}A0^{Arg}-insulin product for an overall yield of approximately 10%.

### EXAMPLE 4

### Acylation of Recombinant Human Insulin with Boc-Arg(Pbf)Arg(Pbf)-NHS Ester in Water/DMF to produce B-1^{Arg}B0^{Arg}-Insulin

Due to the co-elution of the products monoacylated at either the B-chain N terminus or the side chain amine of B29^{Lys} (see example 3 above), the recombinant human insulin was first protected with tert-butyloxycarbonyl (Boc) groups on the A-chain N terminus and the B29^{Lys} side chain amine. Recombinant human insulin (320 mg) was dissolved in 20 mL of 1:1 CH₃CN:PBS buffer, and the pH was adjusted to 10.6. Di-tert-butyl dicarbonate (2.5 equivalents) ((Boc)₂O) was added (55 mg in 270 μL CH₃CN). After 30 min, the pH dropped to approximately 8.7. The pH was adjusted back to approximately 11 with 5 M KOH, and the reaction was then allowed to proceed for an additional 2.5 hr. At this point, LC-MS analysis indicated the presence of three major products with the mass of mono-, di-, and tri-Boc derivatized species, respectively.

The HPLC peak areas indicated that the mono-, di- and tri-Boc derivatives were present in approximately 15:60:25 relative ratios, respectively. Purification of the material was carried out on the C₁₈ preparative column as in Example 1 with a three-stage linear AB gradient of: (1) 0 to 20% B over time range 0 to 20 min; (2) 20 to 25% B over time range 20 to 30 min; and (3) 25 to 75 % B over time range 30 to 230 min. The di-Boc derivatized product (Boc₂-insulin) was obtained after lyophilizing in a yield of 82 mg. LC-MS analysis of the S.aureus V8 protease digest proved conclusively, that the product contained Boc groups on the A-chain N terminus and B29:Lys side chain.

The 82 mg of Boc₂-insulin (0.014 mmol) was dissolved in 10 mL of 1:1 DMF:PBS buffer and the pH was adjusted to approximately 8. Boc-Arg(Pbf)Arg(Pbf)-NHS Ester was prepared as in Example 3 above and dissolved at a concentration of 0.05 mmol/mL in DMF. 1.4 mL of NHS ester solution (0.07 mmol) was added to the Boc₂-insulin and allowed to react for 1 hr. An additional 0.6 mL of Boc-Arg(Pbf)Arg(Pbf)-NHS ester solution (0.03 mmol) was added, and the reaction was continued for another hour. The product was analyzed by analytical HPLC as in Example 1 but using the linear AB gradient of 25 to 100% B over 25 min, in which A=0.05% TFA/H₂O and B=0.05% TFA/CH₃CN and the flow rate was 1 mL/min. It was found that one peak appeared with the correct MW of the Boc-Arg(Pbf)Arg(Pbf)-Boc₂-insulin product.

Purification was carried out with the Vydac C₁₈ column as in Example 1 with the three-stage linear AB gradient of: (1) 0 to 25 % B over time range 0 to 20 min; (2) 25 to 40% B over time range 20 to 40 min; and (3) 40 to 100% B over time range 40 to 100 min. The purified, fully-protected product yielded 36 mig after lyophilization. The material was treated for 1 hr at room temperature with 10 mL of 94:2:2:2 TFA:anisole:MeOH: TIPS to give the fully-deprotected product. The mixture was then concentrated to near dryness and redissolved in 10 mL of 10:90 CH₃CN:H₂O, which was extracted twice with 15 mL diethyl ether. Final reversed-phase HPLC purification (as in Example 1) yielded 24 mg of final B-1^{Arg}B0^{Arg}-insulin product for an overall yield of approximately 8%.

### EXAMPLE 5

### Acylation of A0^{Arg}-Insulin with Boc-Arg(Pbf)Arg(Pbf)-NHS Ester in H₂O/CH₃CN to Produce A0^{Arg}B-1^{Arg}B0^{Arg}-Insulin

Boc-Arg(Pbf)Arg(Pbf)-NHS Ester (0.5 mmol) was prepared as in Example 3 and dissolved in 4 mL MeOH. A0^{Arg}-insulin (320 mg, 0.054 mmol) was dissolved in 40 mL of 1:1 CH₃CN:PBS buffer at pH 10. The pH was reduced to approximately 7. The solution began to get cloudy due to the protein being near its pI of approximately 6.3. Half the Boc-Arg(Pbf)Arg(Pbf)-NHS Ester solution was added (0.25 mmol; approximately 4.7 equiv.) and the solution was sonicated for 15 min then mixed on a rotisserie for 75 min. HPLC analysis indicated two peaks, confirmed by LC-MS to be monoacylated products, present in a ratio of approximately 85:15. The sample was acidified with 100 μL TFA then diluted with 20 mL H₂O. Reversed-phase purification was carried out as in Example 2, and yielded 55 mg of the major mono-acylated product after lyophilization. The peptide was deprotected with 20 mL of the TFA cocktail described in Example 3 for 2 hr, evaporated to near dryness, redissolved in 20 mL of 10:90 CH₃CN:H₂O, and extracted with 20 mL hexane. Final reversed-phase HPLC purification as in Example 1 yielded 38 mg of product (a yield of 12%). This material was subsequently confirmed to be the desired A0^{Arg}B-1^{Arg}B0^{Arg}-insulin.

### EXAMPLE 6

### Acylation of Recombinant Human Insulin with Boc-Arg(Boc)₂-NHS Ester in Water/CH₃CN to produce A0^{Arg}B0^{Arg}B29^{Lys-Nε-Arg}-Insulin

Recombinant human insulin-zinc crystals (307 mg; 0.053 mmol) were dissolved in 30 mL of 1:1 CH₃CN:PBS-buffer. 5M KOH solution was added (50 μL) to dissolve the crystals at pH 10. The pH was then reduced to approximately 7.5 with 5 M phosphoric acid. Boc-Arg(Boc)₂-NHS ester (1 mmol) was prepared as in Example 1 and dissolved in 4 mL MeOH. 2 mL of Boc-Arg(Boc)₂-NHS ester solution (0.5 mmol) was added to the insulin solution and the resulting mixture was mixed at room temperature for 2 hr. The pH at this point had dropped to approximately 6.6. Addition of 50 uL of 5M KOH solution increased the pH back to 7.2.

Then the remaining Boc-Arg(Boc)₂-NHS ester was added to the insulin mixture and the reaction was continued for an additional 3 hr. The mixture was then acidified with 100 μL TFA, diluted with 30 mL of water, and lyophilized overnight. The lyophilized material totaling approximately 1.07 g due to the presence of excess acylating reagent and salts from the PBS buffer was dissolved in 20 mL of TFA and allowed to sit at room temperature for 1.5 hr to give the deprotected product. The mixture was then evaporated to near dryness on a rotary evaporator and redissolved in 20 mL of 1:9 CH₃CN:H₂O.

The sample was analyzed by analytical reversed-phase HPLC as in Example 1 and displayed a similar chromatographic profile with a major peak due to tri-acylated product, and smaller amounts of tetra-acylated and di-acylated insulin eluting just before and just after the main peak, respectively. The relative amounts of the products were not determined since they were not fully resolved under these conditions, but approximately 70% of the material appeared to be the tri-acylated species (as also observed in Example 1).

The crude acylated material was purified by cation exchange chromatography as in Example 1. The combined purified tri-acylated insulin was concentrated from approximately 96 mL to 75 mL, diluted back to I 00 mL with H₂O and loaded on a Vydac C₁₈ preparative column and purified as in Example 1. The combined purified material was lyophilized to give 96 mg (overall yield was approximately 31%).

### EXAMPLE 7

### Acylation of A21^{Gly}-Insulin with Boc-Arg(Boc)₂-NHS Ester in H₂O/CH₃CN to Produce A0^{Arg}B0^{Arg}B29^{Lys-Nε-Arg}A21^{Gly}-Insulin

Lyophilized A21^{Gly}-insulin (65 mg; 0.011 mmol) was dissolved in 8 mL of 1:1 CH₃CN:PBS-buffer. The pH was adjusted to 7.5 with 5 M KOH solution. Boc-Arg(Boc)₂-NHS ester (0.4 mmol) was prepared as in Example 1 and dissolved in 2 mL MeOH. Boc-Arg(Boc)₂-NHS ester solution (1 mL, 0.2 mmol; 17 equivalents) was added to the A21:G-insulin solution, and the resulting mixture was mixed at room temperature for 3 hr. The pH at this point had dropped to approximately 6.4. Addition of 20 μL of 5M KOH solution increased the pH back to 7.5. Then the remaining 0.2 mmol Boc-Arg(Boc)₂-NHS ester was added to the insulin mixture and the reaction was continued for an additional 3 hr. The mixture was then acidified with 50 μL TFA, diluted with 10 mL of water, and lyophilized overnight. The lyophilized material containing peptide, excess acylating reagent, and salts from the PBS buffer was dissolved in 20 mL of TFA and allowed to sit at room temperature for 1 hr to give the deprotected product. The mixture was evaporated to near dryness on a rotary evaporator and redissolved in 20 mL of 1:9 CH₃CN:H₂O, which was then extracted with 20 mL hexane. The sample was analyzed by analytical reversed-phase HPLC as in Example 1 and displayed a similar chromatographic profile with a major peak due to tri-acylated product, and smaller amounts of tetra-acylated and di-acylated insulin eluting just before and just after the main peak, respectively. The relative amounts of the products was not determined since they were not fully resolved under these chromatographic conditions, but approximately 60-70% of the material appeared to be the desired tri-acylated species.

The crude acylated material was purified by cation exchange chromatography as in Example 1. The combined purified tri-acylated insulin was concentrated from approximately 96 mL to 75 mL, diluted back to 100 mL with H₂O and loaded on a Vydac C₁₈ semi-preparative column (10 mm i.d.×250 mm). The sample was eluted with a flow rate of 4 mL/min using a two-stage linear AB gradient of 0 to 25% B over 15 min followed by 25 to 75% B over 100 min, where A=0.05% TFA/H₂O and B=0.05% TFA/CH₃CN. The purified material was lyophilized to give 21 mg (overall yield was approximately 32%).

### EXAMPLE 8

### Acylation of Insulin with Boc-Lys(Boc)-NHS Ester in Water/CH₃CN to Produce A0^{Lys}B0^{Lys-Nε-Lys}-Insulin, and Guanidylation with N,N′-bis-Boc-1-guanylpyrazole (Boc₂-guanylpyrazole) to A0^{gHR}B0^{gHR}B29^{Lys-Nε-gHR}-Insulin

“gHR” means alpha-guanidinyl homoarginine. Recombinant human insulin-Zn crystals (300 mg, 0.052 mmol) were dissolved in 20 mL CH₃CN:PBS buffer 1:1 at pH 10 and the pH was then adjusted to approx. 7. Ten equivalents of Boc-Lys(Boc)-NHS ester was added (230 mg in 2 mL CH₃CN), and the solution was mixed at room temperature for 2 hr. At this time, an additional 230 mg of Boc-Lys(Boc)-NHS ester was added and the reaction was continued for 2.5 hr. LC-MS analysis indicated a large amount of tri-acylated species present and a smaller amount of di-acylated species. The mixture was diluted to 50 mL with H₂O and lyophilized. Deprotection in 20 mL TFA for 1 hr followed by LC-MS indicated that again there was approximately 70% of the insulin in the tri-acylated form, flanked by about 10% tetra-acylated product eluting slightly earlier and 20% di-acylated product eluting slightly after the major product. The sample was evaporated to near dryness, redissolved in 30 mL 30:70 CH₃CN:H₂O, split in two equal portions, and lyophilized. One of the lyophilized portions (0.026 mmol) was dissolved in 10 mL of MeOH:H₂O 9:1, and 0.5 mL of triethylamine was added. The apparent pH was 9.3. Boc₂-guanylpyrazole (160 mg, 0.52 mmol) was added and the reaction was allowed to go 1 hr. An additional 160 mg Boc₂-guanylpyrazole was added and the reaction was continued for another hour. At this point LC-MS analysis indicated the presence of products with one to four Boc₂-guanyl groups added.

An additional 800 mg Boc₂-guanylpyrazole (2.6 mmol) was added, and the reaction was continued for another 4 hr. The total of 3.6 mmol of Boc₂-guanylpyrazole added, less the amount expected to react with the amino groups of the excess Lysine added in the initial acylation step (1 mmol) gives 2.6 mmol available to react with the triLys-insulin (an excess of approximately 15 equiv. of reagent per amino group).

At the end of the 6 hr reaction period, the sample was concentrated on a rotary evaporator to near dryness, redissolved in 10 mL of CH₃CN:H₂O 60:40, and lyophilized. The lyophilized sample was treated with 20 mL TFA for 2 hr to give the deprotected product, concentrated to dryness, and redissolved in 20 mL 15:85 CH₃CN:H₂O. LC-MS analysis at this point showed a main peak (approximately 60% of the material) with the expected mass of the hexa-guanidylated product and also a smaller amount of coeluting penta-guanidylated product.

Purification by cation exchange chromatography was carried out as in Example 1, but using a different linear AB gradient of 25 to 70% B over 100 min with a flow rate of 4 mL/min and 8 mL fractions. Pooled fractions (88 mL total volume) were concentrated on a rotary evaporator to approximately 65 mL then diluted back to 90 mL with H₂O. The sample was subjected to a final RP-HPLC purification as in Example 1, yielding 43 mg of final product (overall yield approximately 29%).

### EXAMPLE 9

### Acylation of Recombinant Human Insulin with Boc-Lys(tfa)-NHS Ester in Water/CH₃CN to Produce A0^{Lys}B0^{Lys}B29^{Lys-Nε-Lys}-Insulin

Recombinant human insulin-Zn crystals (200 mg, 0.034 mmol) were dissolved in 10 mL of 1:1 CH₃CN:H₂O at pH 10, then the pH was adjusted to approximately 7 with 6 M phosphoric acid. Boc-Lys(tfa)-NHS ester (1 mmol) was prepared from Boc-Lys(tfa)-OH, NHS, and DCC as in Example 1 and dissolved in 10 mL MeOH. To the insulin solution was added 1.7 mL of Boc-Lys(tfa)-NHS ester solution (0.17 mmol; 5 equivalents). The mixture was allowed to react for 75 min then acidified with 0.5 mL TFA and diluted to 30 mL. Analytical HPLC and LC-MS confirmed the presence of three monoacylated peaks, two diacylated products and one triacylated product.

Reverse-phase HPLC purification as in Example 2 followed by lyophilization of the separated species yielded the protected products as follows: 33 mg A0^{Lys}B0^{Lys}B29^{Lys-Nε-Lys}-insulin; 36 mg A0^{Lys}B0^{Lys}-insulin; 23 mg B0^{Lys}B29^{Lys-Nε-Lys}-insulin; 12 mg A0^{Lys}-insulin; and 31 mg B0^{Lys}-insulin.

Deprotection was carried out in two steps. First, removal of Boc groups from the Lysine alpha-amino groups was achieved by treatment of each of the five samples with 5 mL TFA for 30 min. The solution was then evaporated to near dryness and residual TFA was removed by blowing nitrogen over the sample tube. Then the TFA groups were removed from the lysine ε-amino groups by addition of 6 mL of 15% NH₄OH/H₂O (v:v) and allowing the sample to stay at room temperature for 3-4 hr. The samples were then diluted to 40 mL with H₂O and acidified with acetic acid (1.5 mL) to pH 4. Samples were submitted to a final purification as in Example 1 and yielded final amounts as follows: 14 mg A0^{Lys}B0^{Lys}B29^{Lys-Nε-Lys}-insulin; 17 mg A0^{Lys}B0^{Lys}-insulin; 8 mg B0^{Lys}B29^{Lys-Nε-Lys}-insulin; 5 mg A0^{Lys}-insulin; and 16 mg B0^{Lys}-insulin.

### EXAMPLE 10

### Acylation of A21^{Gly}-Insulin with Boc-Arg(Pbf)-NHS Ester in Water/CH₃CN to Produce A0^{Arg}A21^{Gly}B29^{Lys-Nε-Arg}-Insulin

A21^{Gly}-insulin (230 mg; 0.040 mmol) was dissolved in 24 mL of 1:1 CH₃CN:water. 200 mg of NaH₂PO₄.H₂O was added. 5 M KOH solution was added (approximately 50 μL) to adjust the pH to 10.5. Boc-Arg(Pbf)-NHS ester was prepared from 0.4 mmol each of Boc-Arg(Pbf)-OH, N-hydroxysuccinimide (NHS), and dicyclohexylcarbodiimide (DCC) mixed together in dichloromethane (DCM) for 30 min. The mixture was then filtered and concentrated to dryness on a rotary evaporator. The resulting 0.4 mmol of Boc-Arg(Pbf)-NHS ester was dissolved in 4 mL MeOH. 1 mL of Boc-Arg(Pbf)-NHS ester solution (0.1 mmol; 2.5 equivalents) was added to the insulin solution, and the mixture was stirred at room temperature for 1 hr. The pH at this point had dropped to approximately 9.8. The pH was further reduced to 9.0 with 6 M H₃PO₄. Another 1 mL of Boc-Arg(Pbf)-NHS ester solution (0.1 mmol; 2.5 equivalents) was added to the insulin solution and the mixture was stirred at room temperature for another 1 hr. At this point the mixture was shown by reversed-phase HPLC (carried out on a Zorbax Eclipse XDB-C8 4.6 mm i.d.×15 cm column with a linear AB gradient of 10 to 100% B over 15 min in which A=0.05% TFA/H₂O and B=0.05% TFA in 60:40 CH₃CN:H₂O with flow rate of 1 mL/min) to comprise primarily a monoacylated and a diacylated product in approximately a 57:43 ratio. Another 0.5 mL of Boc-Arg(Pbf)-NHS ester solution (0.05 mmol; 1.25 equivalents) was added to the insulin solution, and the mixture was stirred for 5 min. A second addition of 0.5 mL of Boc-Arg(Pbf)-NHS ester solution was made, and the mixture was stirred for 10 min, then acidified with trifluoroacetic acid (TFA) to pH 3. The solution was diluted with 20 mL of 50:50 CH₃CN:water and filtered. The final reaction mixture contained the major monoacylated and a diacylated products in a 30:70 ratio, as determined by HPLC peak area from UV detection at 220 nm.

The crude acylated material was purified by reversed-phase HPLC on a Vydac C₁₈ 2.2 cm i.d.×25 cm preparative column. The sample was eluted with a flow rate of 12 mL/min using a two-stage linear AB gradient of: (a) 0 to 18% B over 15 min followed by (b)18 to 68% B over 100 min, where A=0.05% TFA/H₂O and B=0.05% TFA/CH₃CN. The fractions containing the diacylated insulin were pooled and lyophilized to yield 134 mg of protected product. This material was deprotected with a mixture of 20 mL of 91:3:3:3 TFA:anisole:MeOH:triisopropylsilane (TIPS) for 1.5 hr at room temperature, then concentrated to near dryness on a rotary evaporator and redissolved in 25 mL of 10:90 CH₃CN:H₂O, which was extracted twice with 20 mL diethyl ether. Final reversed-phase HPLC purification was performed on the same Vydac C₁₈ column described above at 12 mL/min with a two-stage linear AB gradient of: (a) 0 to 15% B over 15 min followed by (b)15 to 55% B over 100 min, where A=0.05% TFA/H₂O and B=0.05% TFA/CH₃CN. This yielded 77 mg of A0^{Arg}A21^{Gly}B29^{Lys-Nε-Arg}-insulin product for an overall yield of approximately 33%.

### EXAMPLE 11

### Acylation of A21^{Gly}-Insulin with (1) Boc-Arg(Pbf)-NHS Ester and (2) Boc-Lys(Boc-Arg(Pbf))-NHS Ester in Water/CH₃CN to Produce A0^{Lys-Nε-Arg}B29^{Lys-Nε-Arg}A21^{Gly}-Insulin

Boc-Lys(Boc-Arg(Pbf))-OH was synthesized on Cl-(2′-chloro)trityl polystyrene polymer. The polymer was loaded with a two-fold excess of Boc-Lys(Fmoc)-OH in a 90:10 dimethylformamide (DMF):diisopropylethylamine (DIEA) mixture. The Fmoc group was subsequently removed from the Lysine ε-amino group with a 20% solution of piperidine in DMF. The α-carboxyate of Fmoc-Arg(Pbf)-OH (four-fold excess) was then coupled to the free amino group via activation with O-benzotriazole-N,N,N′,N′-tetramethyluronium-hexafluoro-phosphate (HBTU) and DIEA in a ratio of amino acid:HBTU:DIEA of 1:0.95:3 in a DMF solution. The Fmoc group was removed from the ε-amino group of Arg with a 20% solution of piperidine in DMF followed by capping of the free amine with a five-fold excess of Di-tert-butyl-dicarbonate (Boc-anhydride) and DIEA in a ratio of 1:2 in a DMF solution. The compound was cleaved from the polymer by two treatments with 30 mL of 1:2 hexafluoroisopropanol (HFIP):dichloromethane (DCM) for 40 min each. The combined solution was filtered and evaporated on a rotary evaporator. Boc-Arg(Pbf)-NHS ester and Boc-Lys(Boc-Arg(Pbf))-NHS ester were prepared as described in Example 1, mixing equal parts NHS and DCC with the respective acids in DCM.

A21^{Gly}-insulin (230 mg; 0.040 mmol) was dissolved in 24 mL of 1:1 CH₃CN:water. 200 mg of NaH₂PO₄.H₂O was added. 5 M KOH solution was added (approximately 50 μL) to adjust the pH to 10.5. Boc-Arg(Pbf)-NHS ester (0.4 mmol) was dissolved in 4 mL MeOH. 1 mL of Boc-Arg(Pbf)-NHS ester solution (0.1 mmol; 2.5 equivalents) was added to the insulin solution and the mixture was stirred at room temperature for 40 min. At this point the mixture was shown by reversed-phase HPLC (carried out on a Zorbax Eclipse XDB-C8 4.6 mm i.d.×15 cm column with a linear AB gradient of 10 to 100% B over 15 min in which A=0.05% TFA/H₂O and B=0.05% TFA in 60:40 CH₃CN:H₂O with flow rate of 1 mL/min) to comprise primarily the starting material and a monoacylated product in a 40:60 ratio. Another 0.6 mL of Boc-Arg(Pbf)-NHS ester solution (0.06 mmol; 1.5 equivalents) was added to the insulin solution and the mixture was stirred at room temperature for another 15 min, at which point, the insulin was primarily converted to the monoacylated species. The pH at this point was reduced from 10.2 to 9.0 with addition of 6 M H₃PO₄. Boc-Lys(Boc-Arg(Pbf))-NHS ester (0.12 mmol) was dissolved in 2 mL MeOH and added to the insulin solution. The mixture was allowed to stir at room temperature for 30 min, then diluted with 20 mL of 50:50 CH₃CN:water, and acidified with 300 uL TFA and filtered. The major peak observed by reversed-phase HPLC corresponded to the product derivatized with one each of Boc-Arg(Pbf) and Boc-Lys(Boc-Arg(Pbf)) as confirmed by HPLC-mass spectral analysis.

The crude acylated material was purified by reversed-phase HPLC on a Vydac C₁₈ 2.2 cm i.d.×25 cm preparative column. The sample was eluted with a flow rate of 13 mL/min using a two-stage linear AB gradient of: (a) 0 to 30% B over 20 min followed by (b) 30 to 80% B over 100 min, where A=0.05% TFA/H₂O and B=0.05% TFA/CH₃CN. The fractions containing the diacylated insulin were pooled and lyophilized to yield 105 mg of protected product. This material was deprotected with a mixture of 20 mL of 91:3:3:3 TFA:anisole:MeOH:triisopropylsilane (TIPS) for 2 hr at room temperature, then concentrated to near dryness and redissolved in 25 mL of 10:90 CH₃CN:H₂O, which was extracted three times with 20 mL diethyl ether. Final reversed-phase HPLC purification was performed on the same Vydac C₁₈ column described above at 12 mL/min with a two-stage linear AB gradient of: (a)0 to 15% B over 15 min followed by (b) 15 to 55% B over 100 min, where A=0.05% TFA/H₂O and B=0.05% TFA/CH₃CN. This yielded 60 mg of A0^{Lys-Nε-Arg}A21^{Gly}B29^{Lys-Nε-Arg}-insulin product for an overall yield of approximately 25%.

### EXAMPLE 12

### Preparation of A0^{Lys-Nε-Arg}A21^{Gly}B29^{Lys-Nε-Arg}-Insulin

A plasmid containing sequence encoding the human proinsulin analog A21^{Gly}C64^{Arg}C65^{Lys}-human proinsulin was expressed in E. coli. The proinsulin analog was purified and folded, and then acylated as follows. Boc-Arg(Boc)₂-NHS ester was prepared from 0.4 mmol each of Boc-Arg(Boc)₂-OH, N-hydroxysuccinimide (NHS), and dicyclohexylcarbodiimide (DCC) mixed together in 3 mL dichloromethane (DCM) for 40 min. The mixture was then filtered and concentrated to dryness on a rotary evaporator. The resulting 0.4 mmol Boc-Arg(Boc)₂-NHS ester was then dissolved in 4 mL MeOH.

Approximately 108 mg of A21^{Gly}C64^{Arg}C65^{Lys}-human proinsulin in 180 mL of 10 mM HCl solution was split in two equal portions and lyophilized. One of the proinsulin portions was redissolved with 12 mL of 50/50 water/CH₃CN. NaH₂PO₄ (80 mg) was added to give a PO₄ concentration of approximately 50 mM. The pH was adjusted to 8.2 with 5 M KOH solution. One mL of Boc-Arg(Boc)₂-NHS ester (0.1 mmol; approximately 20 equivalents) was added, and the mixture was stirred at room temperature for 2.5 hr, after which time the pH had dropped to 7.4. The pH was adjusted back to 8.2, and another 1 mL of Boc-Arg(Boc)₂-NHS ester solution was added. The solution was mixed for an additional 3 hr, then diluted to 50 mL with water, acidified with 200 uL trifluoroacetic acid (TFA), and lyophilized. The lyophilized reaction mixture was redissolved in 20 mL of 95:5 TFA:water and left at room temperature for 1.5 hr. The TFA mixture was evaporated to near dryness on a rotary evaporator, then diluted with 25 mL of 10% CH₃CN/water and extracted twice with 20 mL diethyl ether. The deprotected mixture was analyzed by reversed-phase HPLC on a Zorbax Eclipse XDB-C8 4.6 mm i.d.×15 cm column with a linear AB gradient of 10 to 100% B over 15 min in which A=0.05% TFA/H₂O and B=0.05% TFA in 60:40 CH₃CN:H₂O with flow rate of 0.9 mL/min and mass spec. detection and found to contain small amounts of “overacylated” product in which more than the expected three additional Arg residues are attached (one each at the N terminal amine and the Lysine side chain amines of B29:Lys and C65:Lys). This is presumably due to attachment of Arg residues at side chain phenolic groups of Tyr, imidazole groups of His side chains or other reactive side chain moieties. The proinsulin solution was increased to pH 10.5 for 30 min with the intention to reduce the amount of overacylation products by base-catalyzed hydrolysis of these bonds. The amount of overacylated species was substantially decreased by this pH excursion process. After the 30 min high pH treatment, the pH was reduced back to approximately 3 with TFA and the solution was stored at −20° C.

The chemical modification, deprotection and pH excursion procedure was repeated for the second portion of A21^{Gly}C64^{Arg}C65^{Lys}-human proinsulin. The resulting solutions of A21^{Gly}B29^{Lys-Nε-Arg}C64^{Arg}C65^{Lys-Nε-Arg}-human proinsulin derivative were combined and lyophilized. The purity of the crude, deprotected material was approximately 65%, as judged by the reversed-phase HPLC peak area.

The acylated proinsulin derivative was digested with trypsin and Carboxypeptidase B to remove the leader sequence and the “C peptide” from residues C31Arg through C64Arg while keeping intact the C65Lys-Nε-Arg and B29Lys-Nε-Arg moieties to form the A0^{Lys-Nε-Arg}A21^{Gly}B29^{Lys-Nε-Arg}-insulin derivative. The formation of the Des-30 insulin product was effectively blocked by the modification on B29^{Lys}. Purified A0^{Lys-Nε-Arg}A21^{Gly}B29^{Lys-Nε-Arg}-insulin was used in in vitro and in vivo experiments, as follows.

### EXAMPLE 13

### In Vitro Receptor Affinity

The affinity of insulin molecules for the human insulin receptor (IR) was measured in a competitive binding assay using radiolabeled ligand, [¹²⁵I] insulin. Human insulin receptor membranes were prepared as P1 membrane preparation of stable transfected 293EBNA cells overexpressing the receptor. The assay was developed and validated in both filtration and SPA (scintillation proximity assay) mode with comparable results, but was performed in the SAP mode employing PVT PEI treated wheatgerm agglutinin-coupled SPA beads, Type A (WGA PVT PEI SPA) beads from Amersham Pharmacia Biotech.

Radiolabeled ligand ([¹²⁵I] recombinant human insulin) was prepared in house or purchased from Amersham Pharmacia Biotech, at specific activity 2000 Ci/mmol on the reference date. SPA assay buffer was 50 mM Tris-HCL, pH 7.8, 150 mM NaCl, 0.1% BSA. The assay was configured for high throughput in 96-well microplates (Costar, #3632) and automated with radioligand, membranes and SPA beads added by Titertec/Plus (ICN Pharmaceuticals).

The reagents were added to the plate wells in the following order:

| Reagent | Final concentration |
|---|---|
| Control or insulin molecule | Min signal (BHI) = 0.1 μM, all other |
| dilution | compounds [Hi] = 0.1 μM |
| [¹²⁵I] recombinant human insulin | 50 pM |
| HIR membranes | 1.25 μg |
| WGA PVT PEI SPA beads | 0.25 mg/well |

The plates were sealed with an adhesive plate cover and shaken for 1 min on LabLine Instruments tier plate shaker. The plates were incubated at room temperature (22° C.) for 12 hours by placing them in a Wallac Microbeta scintillation counter and setting the timer for 12 hours. The counting was done for 1 min per well using protocol normalized for [¹²⁵I].

IC₅₀ for each insulin molecule was determined from 4-parameter logistic non-linear regression analysis. Data was reported as mean±SEM. Relative affinity was determined by comparing each insulin molecule to the recombinant human insulin control within each experiment and then averaging the relative affinity over the number of experiments performed. Therefore, a comparison of the average IC₅₀ for an insulin molecule with the average IC₅₀ for insulin does not generate the same value.

The affinity of each insulin molecule and recombinant human insulin for insulin growth factor receptor (IGF1-R) was measured in the competitive binding assay using [¹²⁵I]IGF-1 radiolabeled ligand. Human IGF-1 receptor membranes were prepared as P1 membrane preparation of stable transfected 293EBNA cells overexpressing the receptor. The assay was developed and validated in both filtration and SPA (scintillation proximity assay) mode with comparable results, but was routinely performed in the SAP mode employing PVT PEI treated wheatgerm agglutinin-coupled SPA beads, Type A (WGA PVT PEI SPA) beads from Amersham Pharmacia Biotech. [¹²⁵I]IGF-1 radiolabeled ligand was prepared in house or purchased from Amersham Pharmacia Biotech, at specific activity 2000 Ci/mmol on the reference date. SPA assay buffer was 50 mM Tris-HCL, pH 7.8, 150 mM NaCl, 0.1% BSA. The assay was configured for high throughput in 96-well microplates (Costar, #3632) and automated with radioligand, membranes and SPA beads added by Titertec/Plus (ICN Pharmaceuticals).

The reagents were added to the plate wells in the following order.

| Reagent | Final concentration |
|---|---|
| Control or insulin molecule | Min signal (IGF-1) = 1 μM, all other |
| dilution | compounds [Hi] = 10 μM |
| [¹²⁵I] IGF-1 | 50 pM |
| IGF-1R membranes | 1.25 μg |
| WGA PVT PEI SPA beads | 0.25 mg/well |

The plates were sealed with adhesive plate cover and shaken for 1 min on LabLine Instruments tier plate shaker. The plates were incubated at room temperature (22° C.) for 12 hours by placing them in a Wallac Microbeta scintillation counter and setting the timer for 12 hours. The counting was done for 1 min per well using protocol normalized for [¹²⁵I].

IC₅₀ for each insulin molecule was determined from 4-parameter logistic non-linear regression analysis. Data was reported as mean±SEM. Relative affinity was determined by comparing each insulin molecule to the recombinant insulin control within each experiment and then averaging the relative affinity over the number of experiments performed. Therefore, a comparison of the average IC₅₀ for each insulin molecule with the average IC₅₀ for insulin does not generate the same value.

The selectivity index was calculated as the ratio of TR relative affinity to IGF-1 R relative affinity. A selectivity index >1 indicates a greater relative selectivity for HIR. A selectivity index <1 indicates a greater relative selectivity for IGF-1R.

Table 1 depicts insulin receptor (IR) affinity, insulin-like growth factor 1 (IGF1-R) receptor affinity, and a receptor selectivity index (IR/IGF1-R) for each insulin molecule and recombinant human insulin.

TABLE 1

| | Relative | | | Relative 1GF1-R | | | |
|---|---|---|---|---|---|---|---|
| | IR Affinity | | | Affinity | | | |
| Molecule | Mean | SEM | n | Mean | SEM | n | Index |
| recombinant human insulin | 1.00 | 0.00 | 63 | 1.00 | 0.00 | 63 | 1.00 |
| A0^{Arg}B0^{Arg}B29^{Lys-Nε-Arg}-insulin | 0.60 | 0.06 | 10 | 0.84 | 0.03 | 9 | 0.71 |
| A0^{Arg}A21^{Gly}B29^{Lys-Nε-Arg}-insulin | 0.34 | 0.02 | 8 | 0.39 | 0.03 | 8 | 0.87 |
| A0^{Lys-Nε-Arg}A21^{Gly}B29^{Lys-Nε-Arg}-insulin | 0.41 | 0.04 | 8 | 0.4 | 0.04 | 8 | 1.01 |

### EXAMPLE 14

### In Vitro Metabolic Potency

Metabolic potency (glucose uptake) of each insulin molecule and recombinant human insulin was determined in the glucose-uptake assay using differentiated mouse 3T3-L1 adipocytes. Undifferentiated mouse 3T3 cells were plated at density 25,000 cells/well in 100 μl of growth media (DMEM, high glucose, w/out L-glutamine, 10% calf serum, 2 mM L-glutamine, 1% antibiotic/antimycotic solution).

Differentiation was initiated 3 days after plating by addition of differentiation media: DMEM, high-glucose, w/out L glutamine, 10% FBS, 2 mM L-Glutamine, 1% antibiotic/antimycotic solution, 10 mM HEPES, 0.25 mM dexamethasone, 0.5 mM 3-isobutyl-1-methylxanthine(IBMX), 5 mg/ml insulin. After 48 hours (day 3), the differentiation media was changed to one with insulin, but without IBMX or dexamethasone and at day 6 the cells were switched to differentiation media containing no insulin, IBMX or dexamethasone. The cells were maintained in FBS media, with changes every other day.

Glucose transport assay was performed using Cytostar T 96 well plates. 24 hours prior to assay cells were switched to 100 μl of serum free media containing 0.1% of BSA. On the day of the assay, the media was removed and 50 μl of assay buffer was added: a so-called KRBH or Krebs-Ringer buffer containing HEPES, pH 7.4 (118 mM NaCL, 4.8 mM KCl, 1.2 mM MgSO₄×7 H₂0, 1.3 mM CaCl₂H₂0, 1.2 mM KH₂PO₄, 15 mM HEPES). Insulin dilutions were prepared in same buffer with 0.1% BSA, and added as 2×. The blank contained KRBH, 0.1% BSA and 20 mM 2×2-deoxy-D-Glucose, 0.2 μCi/well of 2-deoxy-D-(U-¹⁴C) glucose and 2×10⁻⁷ insulin. The cells were incubated at 37° C. for 1 hour. After that period 10 μl of cytochalasin B was added to a final concentration of 200 μM in KRBH, and the plates were read on a Microbeta plate reader. Relative affinity was determined by comparing each insulin molecule to the recombinant human insulin control within each experiment and then averaging the relative affinity over the number of experiments performed. Therefore, a comparison of the average EC₅₀ for each insulin molecule the average EC₅₀ for insulin does not generate the same value.

Table 2 depicts the in vitro metabolic potency for each insulin molecule and recombinant human insulin.

TABLE 2

Metabolic
Potency

| Molecule | Mean | N |
|---|---|---|
| recombinant human insulin | 1.00 | 48 |
| A0^{Arg}B0^{Arg}B29^{Lys-Nε-Arg}-insulin | 0.85 | 4 |
| A0^{Arg}A21^{Gly}B29^{Lys-Nε-Arg}-insulin | 0.48 | 2 |
| A0^{Lys-Nε-Arg}A21^{Gly}B29^{Lys-Nε-Arg}-insulin | 0.4 | 2 |

### EXAMPLE 15

### In Vitro Mitogenicity

The mitogenic potency of each insulin molecule was determined by measuring proliferation of human mammary epithelial cells (HMEC) in culture. HMEC were obtained from Clonetics Corporation (San Diego, Calif.) at passage 7 and were expanded and frozen at passage 8. A fresh ampoule was used for each time so that all experiments were conducted with the same passage 10 of HMEC. Cells were maintained in culture according to Bio Whittaker instructions. To maintain the cell culture, the growth medium was changed every other day and the cultures were inspected daily.

Two products from BioWhittaker were used as the growth medium:
1. Fully supplemented MEGM (CC-3051), including: (amounts indicate final concentrations, except BPE)
10 ng/ml hEGF (human recombinant Epidermal Growth Factor)
5 μg/ml Insulin
0.5 μg/ml Hydrocortisone
50 μg/ml Gentamicin, 50 ng/ml Amphotericin-B
13 mg/ml BPE (Bovine pituitary Extract) 2 ml (attached); and
2. Basal Medium (MEBM, CC-3151) with all the supplements listed below (SingleQuots, CC-3150)
13 mg/ml BPE (Bovine Pituitary Extract (CC-4009) 2 ml
10 pg/ml hEGF (CC-4017) 0.5 ml
5 μg/ml Insulin (CC-4031) 0.5 ml
0.5 mg/ml Hydrocortisone (CC-4031) 0.5 ml
50 mg/ml Gentamicin, 50 mg/ml Amphotericin-B (CC-4081) 0.5 ml.

For a growth experiment, the assay medium was growth medium without 5 μg/ml Insulin, and with 0.1% BSA. The assay was performed in 96 well Cytostart scintillating microplates (Amersham Pharmacia Biotech, RPNQ0162). Recombinant human insulin and IGF-1 were controls used in each assay run, and recombinant human insulin was on each assay plate.

The assays were performed according to the following protocol. On day one, HMECs were seeded at a density of 4000 cells/well in 100 μl of Assay Medium. Insulin in the growth medium was replaced with graded doses of recombinant human insulin or an other insulin molecule from 0 to 1000 nM final concentration. After 4-hour incubation, 0.1 μCi of ¹⁴C-thymidine in 10 μl of assay medium was added to each well and plates were read at 48 h and/or 72 h on Trilux.

Typically, the maximal growth response was between 3-4-fold stimulation over basal. Response data were normalized to between 0 and 100% response equal to 100×(response at concentration X−response at concentration zero) divided by (response at maximal concentration−response at zero concentration). Concentration-response data were fit by non-linear regression employing JMP software.

Relative mitogenic potency was determined by comparing each insulin molecule to insulin control within each experiment and then averaging the relative potency over the number of experiments performed. Therefore, a comparison of the average EC₅₀ for each insulin molecule with the average EC₅₀ for insulin does not generate the same value.

Table 3 depicts the in vitro mitogenicity, measured in terms of cell proliferation, for each insulin molecule. The data in Table 3 show that each of insulin molecules is less mitogenic than recombinant human insulin.

TABLE 3

| | Mitogenic Potency | | |
|---|---|---|---|
| Molecule | Mean | SEM | N |
| recombinant human insulin | 1.00 | 0.00 | 250 |
| A0^{Arg}B0^{Arg}B29^{Lys-Nε-Arg}-insulin | 0.76 | 0.10 | 4 |
| A0^{Arg}A21^{Gly}B29^{Lys-Nε-Arg}-insulin | 0.35 | 0.04 | 7 |
| A0^{Lys-Nε-Arg}A21^{Gly}B29^{Lys-Nε-Arg}-insulin | 0.36 | 0.03 | 7 |

### EXAMPLE 16

### Phosphate Buffered Saline Solubility

An in vitro precipitation assay that is indicative of a propensity to extend time-action in vivo was developed as follows. An aqueous solution adjusted to pH 4 and containing a pharmacological dose (100 international units) of an insulin molecule and 30 μg/ml of Zn²⁺, 2.7 mg/ml of m-cresol and 17 mg/ml glycerol % wvas neutralized with phosphate buffered saline (PBS) to 2 international units and centrifuged for 5 min at 14,000 rpm and RT. The supernatant was removed and approximately one tenth of the supernatant was injected into an analytical Symmetry Shield RP8 RP-HPLC system (Waters, Inc.). Area under the eluted peak was integrated and compared to area under the peak of reference standard, which was either recombinant human insulin in 0.1N HCl. The ratio of the areas was multiplied by 100 to generate % solubility in PBS.

The PBS solubility for the recombinant human insulin formulation and for each insulin molecule is shown in Table 4.

TABLE 4

| Molecule | PBS Solubility |
|---|---|
| recombinant human insulin | 89.5 |
| A0^{Arg}B0^{Arg}B29^{Lys-Nε-Arg}-insulin | 22.4 |
| A0^{Arg}A21^{Gly}B29^{Lys-Nε-Arg}-insulin | 19.1 |
| A0^{Lys-Nε-Arg}A21^{Gly}B29^{Lys-Nε-Arg}-insulin | 12.9 |

### EXAMPLE 17

### Isoelectric Point

Isoelectric focusing is an electrophoretic technique that separates proteins on the basis of their isoelectric points (pI). The pI is the pH at which a protein has no net charge and does not move in in electric field. IEF gels effectively create a pH gradient so proteins separate on their unique pI property. Detection of protein bands can be accomplished by sensitive dye staining like Novex Collodial Coomassie Staining Kit. Alternatively, detection can be achieved by blotting the gel onto polyvinylidene difluoride (PVDF) membrane and staining it with Ponceau Red. The pI of a protein is determined by comparing it to pI of a known standard. IEF protein standards are combination of proteins with well-characterized pI values blended to give uniform staining. Yet another method of pI determination is IEF by capillary electrohoresis (cIEF). The pI is determined by comparison to known markers.

The isoelectric point (pI) of recombinant human insulin and each insulin molecule was determined by isoelectric focusing gel electrophoresis using Novex IEF gels of pH 3-10 that offer pI performance range of 3.5-8.5. The isoelectric points are shown in Table 5.

TABLE 5

| Molecule | Isoelectric Point |
|---|---|
| recombinant human insulin | 5.62 |
| A0^{Arg}B0^{Arg}B29^{Lys-Nε-Arg}-insulin | 7.15 |
| A0^{Arg}A21^{Gly}B29^{Lys-Nε-Arg}-insulin | 6.80 |
| A0^{Lys-Nε-Arg}A21^{Gly}B29^{Lys-Nε-Arg}-insulin | 7.10 |

### EXAMPLE 18

### In Vivo Study in Dogs

Experiments were conducted in overnight-fasted, chronically cannulated (femoral artery and vein), conscious male and female beagles (Marshall Farms, North Rose, N.Y.). On the day of the experiment, indwelling vascular access ports (Access Technologies, Norfolk Medical, Skokie, Ill.) were accessed and cleared and the animals were placed in 3′×3′ study cages. Dogs were allowed at least 15 minutes to acclimate to the cage environment before an arterial blood sample was drawn for determination of fasting insulin and glucose concentrations (time=−30 minutes). At this time a continuous venous infusion (0.65 μg/kg/min) of cyclic somatostatin (BACHEM, Torrence, Calif.) was initiated and continued for the next 24.5 hours. Thirty minutes after the start of the infusion (time=0), an arterial sample was drawn and a subcutaneous bolus of saline or an insulin preparation (2 nmol/kg) was injected into the dorsal aspect of the neck. Arterial blood samples were taken periodically thereafter for the determination of plasma glucose and insulin concentrations.

Plasma glucose concentrations were determined the day of the study using a glucose oxidase method in a Beckman Glucose Analyzer II (Beckman Instruments Inc., Brea, Calif.). Plasma samples were stored at −80° C. until time for insulin analysis. Insulin concentrations were determined using commercially available radioimmunoassay kits sensitive to human insulin and insulin molecules.

A0^{Arg}B0^{Arg}B29^{Lys-Nε-Arg}-insulin and NPH insulin each exhibited a time action that was better than the saline control. The A0^{Arg}B0^{Arg}B29^{Lys-Nε-Arg}-insulin solution exhibited a time action comparable to NPH insulin.

A0^{Arg}A21^{Gly}B29^{Lys-Nε-Arg}-insulin and A0^{Lys-Nε-Arg}A21^{Gly}B29^{Lys-Nε-Arg}-insulin were compared to saline and to insulin glargine (A21^{Gly}B31^{Arg}B32^{Arg}-insulin). In each of two studies, A0^{Arg}A21^{Gly}B29^{Lys-Nε-Arg}-insulin, A0^{Lys-Nε-Arg}A21^{Gly}B29^{Lys-Nε-Arg}-insulin, and glargine exhibited a time action that was longer than the saline control. In the first study, A0^{Arg}A21^{Gly}B29^{Lys-Nε-Arg}-insulin and A0^{Lys-Nε-Arg}A21^{Gly}B29^{Lys-Nε-Arg}-insulin exhibited a time action comparable to glargine. In the second study, A0^{Arg}A21^{Gly}B29^{Lys-Nε-Arg}-insulin and A0^{Lys-Nε-Arg}A21^{Gly}B29^{Lys-Nε-Arg}-insulin exhibited a time action that was shorter than glargine.

### EXAMPLE 19

### In Vivo Study in Rats

Experiments were conducted in chronically cannulated (femoral artery and vein), male Sprague Dawley rats after an over-night fast. On the morning of the experiment, the contents of the catheters were aspirated; the ends of the catheters were attached to extension lines; and tile animals were placed in 12″×12″ study cages. After a 30 minute acclimation period, an arterial blood sample was drawn, and an iv bolus of vehicle (saline 2.0 containing 0.3% rat albumin) or insulin molecule (insulin molecule formulation diluted in saline containing 0.3% rat albumin; 0.1, 0.2, 0.4, 0.8, or 1.2 nmol/kg; n=5/dose) was administered. Blood was drawn 10, 20, 30, 45, and 60 minutes after the intravenous injection.

All blood samples were collected into tubes containing disodium EDTA and placed on ice. Samples were centrifuged; tile plasma was collected; and plasma glucose concentrations were determined the day of the study using a Monarch Clinical Chemistry Analyzer.

Area under the glucose curves (0-30 minutes) were calculated using the trapezoidal rule. Resulting values for various doses were graphed using GraphPad Prism. The dose which corresponded to a glucose area under the curve of 2.45 g·min/dL was determined and was used to directly compare the relative potencies of the insulin preparations.

In one experiment, the estimated potency for recombinant human insulin was 0.160 nmol/kg, and was 0.158 nmol/kg for A0^{Arg}B0^{Arg}B29^{Lys-Nε-Arg}-insulin.

In another experiment, the estimated potency for recombinant human insulin was 0.162 nmol/kg, and was 0.200 nmol/kg for A0^{Arg}A21^{Gly}B0^{Arg}B29^{Lys-Nε-Arg}-insulin.

In another experiment, the estimated potency for recombinant human insulin was 0.207 nmol/kg, the estimated potency for A0^{Arg}A21^{Ser}B0^{Arg}B29^{Lys-Nε-Arg}-insulin was 0.226 nmol/kg and the estimated potency for A0^{Arg}A21^{Gly}B29^{Lys-Nε-Arg}-insulin was 0.268 nmol/kg.

In another experiment, the estimated potency for recombinant human insulin was 0.317 nmol/kg, and the estimated potency for A0^{Lys-Nε-Arg}A21^{Gly}B29^{Lys-Nε-Arg}-insulin was 0.320 nmol/kg.

In another experiment, the estimated potency for recombinant human insulin was 0.217 nmol/kg, the estimated potency for A0^{Lys-Nε-Arg}A21^{Gly}B29^{Lys-Nε-Arg}-insulin was 0.275 nmol/kg, and the estimated potency for A0^{Arg}A21^{Gly}B29^{Lys-Nε-Arg}-insulin was 0.258 nmol/kg.

### EXAMPLE 20

### A0^{Arg}B0^{Arg}-Insulin Zinc Crystals and Protamine-Zinc Crystals

A stock solution A was prepared by dissolving 16.1 g of synthetic glycerin, 0.73 g of phenol and 1.6 mL of m-cresol in approximately 350 mL of sterile water for irrigation. After dissolution, sterile water was added to a final solution weight of 503 g. A protamine sulfate stock solution was prepared by dissolving 0.0366 g of protamine sulfate in 10 mL of sterile water. An A0^{Arg}B0^{Arg}-insulin stock solution was prepared by dissolving 0.0121 g of A0^{Arg}B0^{Arg}-insulin in 1.28 mL of stock solution A. A zinc oxide stock solution was prepared by diluting 1 mL of a 25 mg/mL zinc oxide solution to a final volume of 25 mL, to obtain a final zinc oxide concentration of 1 mg/mL. A sodium phosphate stock solution was prepared by dissolving 0.0577 g of dibasic sodium phoshphate in 15 mL of sterile water. A sodium chloride stock solution was prepared by dissolving 1.1607 g of sodium chloride in 10 mL of sterile water.

For A0^{Arg}B0^{Arg}-insulin zinc crystal experiments, A0^{Arg}B0^{Arg}-insulin, zinc oxide, and stock solution A were mixed at acidic pH. Sodium chloride was also added to some of the samples. All samples were combined to a final volume of 0.1 mL. 0.1 mL of sodium phosphate stock solution was added, and a precipitate was formed. The final pH was adjusted to between 7.4 and 9.3. A0^{Arg}B0^{Arg}-insulin protamine-zinc crystals were prepared the same way, except that protamine sulfate was also combined with A0^{Arg}B0^{Arg}-insulin, zinc oxide, sodium chloride, and stock solution A.

Each sample was then split into two halves. One sample was incubated at 30° C. and the other sample was left at room temperature. The conditions tested are shown in Table 6, and crystals were observed for each set of conditions tested. All concentrations are nominal.

TABLE 6

| | Protamine | | | | |
|---|---|---|---|---|---|
| A0^{Arg}B0^{Arg}-insulin | sulphate | Zinc | NaCl | | |
| (mg/mL) | (mg/mL) | mcg/mL | (mM) | pH | Temp |
| 3.5 | 0 | 0.25 | 0 | 7.4 | RT |
| 3.5 | 0 | 0.25 | 100 | 7.5 | RT |
| 3.5 | 0 | 0.25 | 0 | 8.5 | RT |
| 3.5 | 0 | 0.25 | 100 | 8.5 | RT |
| 3.5 | 0 | 0.25 | 0 | 9.3 | RT |
| 3.5 | 0 | 0.25 | 100 | 9.2 | RT |
| 3.5 | 0.37 | 0.25 | 100 | 7.4 | RT |
| 3.5 | 0.37 | 0.25 | 100 | 8.5 | RT |
| 3.5 | 0.37 | 0.25 | 100 | 9.2 | RT |
| 3.5 | 0 | 0.25 | 0 | 7.4 | 30° C. |
| 3.5 | 0 | 0.25 | 100 | 7.5 | 30° C. |
| 3.5 | 0 | 0.25 | 0 | 8.5 | 30° C. |
| 3.5 | 0 | 0.25 | 100 | 8.5 | 30° C. |
| 3.5 | 0 | 0.25 | 0 | 9.3 | 30° C. |
| 3.5 | 0 | 0.25 | 100 | 9.2 | 30° C. |
| 3.5 | 0.37 | 0.25 | 100 | 7.4 | 30° C. |
| 3.5 | 0.37 | 0.25 | 100 | 8.5 | 30° C. |
| 3.5 | 0.37 | 0.25 | 100 | 9.2 | 30° C. |

### EXAMPLE 21

### A0^{Arg}B0^{Arg}B29^{Lys-Nε-Arg}-Insulin Zinc Crystals and Protamine-Zinc Crystals

A stock solution A and stock solutions of zinc oxide, sodium phosphate, and sodium chloride were prepared as in Example 20.

A protamine sulfate stock solution was prepared by dissovling 0.0332 g of protamine sulfate was dissolved in 10 mL of stock solution A. An A0^{Arg}B0^{Arg}B29^{Lys-Nε-Arg}-insulin stock solution was prepared by dissovling 0.0112 g of A0^{Arg}B0^{Arg}B29^{Lys-Nε-Arg}-insulin in 1.25 mL of stock solution A.

For A0^{Arg}B0^{Arg}B29^{Lys-Nε-Arg}-insulin zinc crystal experiments, A0^{Arg}B0^{Arg}B29^{Lys-Nε-Arg}-insulin, zinc oxide, and stock solution A were mixed at acidic pH. Sodium chloride was also added to some of the samples. All samples were combined to a final volume of 0.1 mL to yield different conditions. 0.1 mL of sodium phosphate stock solution was added, and a precipitate was formed. The final pH was adjusted to between 7.4 and 9.3.

A0^{Arg}B0^{Arg}B29^{Lys-Nε-Arg}-insulin protamine-zinc crystals were prepared the same way, except that protamine sulfate was also combined with A0^{Arg}B0^{Arg}B29^{Lys-Nε-Arg}-insulin, zinc oxide, sodium chloride, and stock solution A.

Each sample was then split into two halves. One sample was incubated at 30° C. and the other sample was left at room temperature. The conditions tested are shown in Table 7, and crystals were observed for each set of conditions tested. All concentrations are nominal.

Further experiments were performed to optimize sodium chloride concentration and pH. A stock solution A was prepared by dissolving 12.8 g of synthetic glycerin, 0.59 g of phenol and 1.28 g of m-cresol in approximately 300 g of sterile water. After dissolution, sterile water for irrigation was added to a final total solution weight of 403 g. A protamine sulfate stock solution was prepared by dissovling 0.033 g of protamine sulfate in 10 mL of stock solution A. An A0^{Arg}B0^{Arg}B29^{Lys-Nε-Arg}-insulin stock solution was prepared by dissolving 0.0042 g of A0^{Arg}B0^{Arg}B29^{Lys-Nε-Arg}-insulin in 0.3 mL of stock solution A. A zinc oxide stock solution was prepared by dissolving 0.0308 g of zinc oxide in 1 mL of 5 N hydrochloric acid, and sterile water was added to a final volume of 25 mL. A sodium phosphate stock solution was prepared by dissolving 0.1893 g of dibasic sodium phosphate in sterile water for irrigation to a final solution volume of 50 mL. A sodium chloride stock solution was prepared by dissolving 1.173 g of sodium chloride in 10 mL of sterile water for irrigation.

A0^{Arg}B0^{Arg}B29^{Lys-Nε-Arg}-insulin, protamine sulfate, zinc oxide, sodium chloride and stock solution A were combined to a final volume of 0.1 mL. 0.1 mL of sodium phosphate stock solution was added, and a precipitate was formed. The final pH was adjusted to between 7.4 and 9.3.

Each sample was then split into two halves. One sample was incubated at 30° C. and the other sample was left at room temperature. The conditions tested are shown in Table 8, and crystals were observed for each set of conditions tested. All concentrations are nominal.

TABLE 7

| A0^{Arg}B0^{Arg}B29^{Lys-Nε-Arg}- | Protamine | | | | |
|---|---|---|---|---|---|
| insulin | sulphate | Zinc | NaCl | | |
| (mg/mL) | (mg/mL) | (mcg/mL) | (mM) | pH | Temp |
| 3.4 | 0 | 0.25 | 0 | 7.4 | RT |
| 3.4 | 0 | 0.25 | 100 | 7.4 | RT |
| 3.4 | 0 | 0.25 | 0 | 8.5 | RT |
| 3.4 | 0 | 0.25 | 100 | 8.5 | RT |
| 3.4 | 0 | 0.25 | 0 | 9.2 | RT |
| 3.4 | 0 | 0.25 | 100 | 9.2 | RT |
| 3.4 | 0.33 | 0.25 | 100 | 7.4 | RT |
| 3.4 | 0.33 | 0.25 | 100 | 8.6 | RT |
| 3.4 | 0.3 | 0.25 | 100 | 9.2 | RT |
| 3.4 | 0 | 0.25 | 0 | 7.4 | 30° C. |
| 3.4 | 0 | 0.25 | 100 | 7.4 | 30° C. |
| 3.4 | 0 | 0.25 | 0 | 8.5 | 30° C. |
| 3.4 | 0 | 0.25 | 100 | 8.5 | 30° C. |
| 3.4 | 0 | 0.25 | 0 | 9.2 | 30° C. |
| 3.4 | 0 | 0.25 | 100 | 9.2 | 30° C. |
| 3.4 | 0.33 | 0.25 | 100 | 7.4 | 30° C. |
| 3.4 | 0.33 | 0.25 | 100 | 8.6 | 30° C. |
| 3.4 | 0.33 | 0.25 | 100 | 9.2 | 30° C. |

TABLE 8

| A0^{Arg}B0^{Arg}B29^{Lys-Nε-Arg}- | Protamine | | | | |
|---|---|---|---|---|---|
| insulin | sulphate | Zinc | NaCl | | |
| (mg/mL) | (mg/mL) | (mcg/mL) | (mM) | pH | Temp |
| 3.5 | 0.33 | 0.25 | 200 | 7.4 | RT |
| 3.5 | 0.33 | 0.25 | 50 | 8.5 | RT |
| 3.5 | 0.33 | 0.25 | 100 | 8.4 | RT |
| 3.5 | 0.33 | 0.25 | 200 | 8.5 | RT |
| 3.5 | 0.33 | 0.25 | 50 | 8.4 | RT |
| 3.5 | 0.33 | 0.25 | 200 | 9.2 | RT |
| 3.5 | 0.33 | 0.25 | 200 | 7.4 | 30° C. |
| 3.5 | 0.33 | 0.25 | 50 | 8.5 | 30° C. |
| 3.5 | 0.33 | 0.25 | 100 | 8.4 | 30° C. |
| 3.5 | 0.33 | 0.25 | 200 | 8.5 | 30° C. |
| 3.5 | 0.33 | 0.25 | 50 | 8.4 | 30° C. |
| 3.5 | 0.33 | 0.25 | 200 | 9.2 | 30° C. |

### EXAMPLE 22

### A0^{Lys-Nε-Arg}A21^{Gly}B29^{Lys-Nε-Arg}-Insulin Zinc Crystals

For the following experiments,.a stock solution A and stock solutions of sodium chloride, sodium phosphate, zinc oxide and sodium citrate were prepared as follows.

A stock solution A was prepared by dissolving 128.2 g of synthetic glycerin, 5.9 g of phenol, 12.9 g of m-cresol and 30.3 g of dibasic sodium phosphate in approximately 3500 mL of milli-Q water. After dissolution, milli-Q water was added to a final solution weight of 4000 g.

A sodium chloride stock solution was prepared by dissolving 1.1614 g of sodium chloride in 10 mL of sterile water for irrigation.

A sodium phosphate stock solution was prepared by dissolving 0.7538 g of dibasic sodium phosphate in 10 mL of sterile water. 0.5 mL of this phosphate solution was diluted into 9.5 mL of sterile water.

A zinc oxide stock solution was prepared by dissolving 0.4 mL of a 25 mg/mL zinc oxide stock solution into 9.6 mL of sterile water, to obtain a final zinc oxide concentration of 1 mg/mL.

A sodium citrate stock solution was prepared by dissolving 2.9597 g of sodium citrate in 10 mL of sterile water.

In one experiment, a stock solution of A0^{Lys-Nε-Arg}A21^{Gly}B29^{Lys-Nε-Arg}-insulin was prepared by dissolving 0.00335 g of A0^{Lys-Nε-Arg}A21^{Gly}B29^{Lys-Nε-Arg}-insulin in 0.65 mL of stock solution A. The solution was cloudy and the pH was approximately 7.1. pH was adjusted to approximately 3.7 to clear the solution.

Crystallization was set up by first combining the A0^{Lys-Nε-Arg}A21^{Gly}B29^{Lys-Nε-Arg}-insulin with zinc oxide, adding stock solution A and sodium chloride stock solution. The pH of the solution was kept below 4. Sodium phosphate stock solution was then added, and a precipitate was formed. The final pH was adjusted to between 6.5 and 9.5. Each sample was then split into three portions. One sample was incubated at 5° C., one at 30° C. and the other sample was left at room temperature. The tested conditions and observations are shown in Table 9. All concentrations are nominal.

TABLE 9

| A0^{Lys-Nε-Arg}A21^{Gly}B29^{Lys-Nε-Arg}- | | | | | | |
|---|---|---|---|---|---|---|
| insulin | Zinc | NaCl | Na₂PO₄ | | | Crystals |
| (mg/mL) | (mcg/mL) | (mM) | (mM) | pH | Temp | Observed |
| 2.6 | 25 | 0 | 21 | 6.6 | 5° C. | No |
| 2.6 | 25 | 0 | 21 | 7.3 | 5° C. | No |
| 2.6 | 25 | 0 | 21 | 8.0 | 5° C. | No |
| 2.6 | 100 | 0 | 21 | 6.4 | 5° C. | No |
| 2.6 | 100 | 0 | 21 | 7.3 | 5° C. | No |
| 2.6 | 100 | 0 | 21 | 8.2 | 5° C. | No |
| 2.6 | 25 | 100 | 21 | 6.5 | 5° C. | No |
| 2.6 | 25 | 100 | 21 | 7.2 | 5° C. | No |
| 2.6 | 25 | 100 | 21 | 8.5 | 5° C. | Yes |
| 2.6 | 100 | 100 | 21 | 6.4 | 5° C. | No |
| 2.6 | 100 | 100 | 21 | 7.2 | 5° C. | No |
| 2.6 | 100 | 100 | 21 | 8.4 | 5° C. | Yes |
| 2.6 | 25 | 0 | 21 | 6.6 | RT | No |
| 2.6 | 25 | 0 | 21 | 7.3 | RT | No |
| 2.6 | 25 | 0 | 21 | 8.0 | RT | No |
| 2.6 | 100 | 0 | 21 | 6.4 | RT | No |
| 2.6 | 100 | 0 | 21 | 7.3 | RT | No |
| 2.6 | 100 | 0 | 21 | 8.2 | RT | No |
| 2.6 | 25 | 100 | 21 | 6.5 | RT | No |
| 2.6 | 25 | 100 | 21 | 7.2 | RT | Yes |
| 2.6 | 25 | 100 | 21 | 8.5 | RT | No |
| 2.6 | 100 | 100 | 21 | 6.4 | RT | No |
| 2.6 | 100 | 100 | 21 | 7.2 | RT | Yes |
| 2.6 | 100 | 100 | 21 | 8.4 | RT | Yes |
| 2.6 | 25 | 0 | 21 | 6.6 | 30° C. | No |
| 2.6 | 25 | 0 | 21 | 7.3 | 30° C. | No |
| 2.6 | 25 | 0 | 21 | 8.0 | 30° C. | Yes |
| 2.6 | 100 | 0 | 21 | 6.4 | 30° C. | No |
| 2.6 | 100 | 0 | 21 | 7.3 | 30° C. | No |
| 2.6 | 100 | 0 | 21 | 8.2 | 30° C. | No |
| 2.6 | 25 | 100 | 21 | 6.5 | 30° C. | Yes |
| 2.6 | 25 | 100 | 21 | 7.2 | 30° C. | Yes |
| 2.6 | 25 | 100 | 21 | 8.5 | 30° C. | No |
| 2.6 | 100 | 100 | 21 | 6.4 | 30° C. | No |
| 2.6 | 100 | 100 | 21 | 7.2 | 30° C. | Yes |
| 2.6 | 100 | 100 | 21 | 8.4 | 30° C. | Yes |

In another experiment, a stock solution of A0^{Lys-Nε-Arg}A21^{Gly}B29^{Lys-Nε-Arg}-insulin was prepared by dissolving 0.0032 g of A0^{Lys-Nε-Arg}A21^{Gly}B29^{Lys-Nε-Arg}-insulin in 0.65 mL of stock solution A. The solution was cloudy and the pH was approximately 7.1. pH was adjusted to approximately 3.7 to clear the solution.

Crystallization was set up by first combining the A0^{Lys-Nε-Arg}A21^{Gly}B29^{Lys-Nε-Arg}-insulin with zinc oxide, adding stock solution A, sodium chloride and/or sodium citrate stock solution. pH of the solution was kept below 4. Sodium phosphate stock solution was then added, and a precipitate was formed. The final pH was adjusted to between 6.5 and 9.5. Each sample was then split into three portions. One sample was incubated at 5° C., one at 30° C. and the other sample was left at room temperature. The tested conditions and observations are shown in Table 10. All concentrations are nominal.

TABLE 10

| A0^{Lys-Nε-Arg}A21^{Gly}B29^{Lys-Nε-Arg}- | | | Na | | | | |
|---|---|---|---|---|---|---|---|
| insulin | Zinc | NaCl | Citrate | Na₂PO₄ | | | Crystals |
| (mg/mL) | (mcg/mL) | (mM) | (mM) | (mM) | pH | Temp | Observed |
| 2.5 | 25 | 0 | 100 | 21 | 6.3 | 5° C. | No |
| 2.5 | 25 | 0 | 100 | 21 | 8.2 | 5° C. | Yes |
| 2.5 | 25 | 0 | 100 | 21 | 7.4 | 5° C. | No |
| 2.5 | 100 | 0 | 100 | 21 | 6.5 | 5° C. | No |
| 2.5 | 100 | 0 | 100 | 21 | 7.5 | 5° C. | Yes |
| 2.5 | 100 | 0 | 100 | 21 | 8.5 | 5° C. | No |
| 2.5 | 25 | 50 | 75 | 21 | 6.5 | 5° C. | No |
| 2.5 | 25 | 50 | 75 | 21 | 7.5 | 5° C. | Yes |
| 2.5 | 25 | 50 | 75 | 21 | 8.3 | 5° C. | Yes |
| 2.5 | 100 | 50 | 75 | 21 | 6.5 | 5° C. | No |
| 2.5 | 100 | 50 | 75 | 21 | 7.5 | 5° C. | Yes |
| 2.5 | 100 | 50 | 75 | 21 | 8.4 | 5° C. | Yes |
| 2.5 | 25 | 0 | 100 | 21 | 6.3 | RT | No |
| 2.5 | 25 | 0 | 100 | 21 | 8.2 | RT | Yes |
| 2.5 | 25 | 0 | 100 | 21 | 7.4 | RT | No |
| 2.5 | 100 | 0 | 100 | 21 | 6.5 | RT | No |
| 2.5 | 100 | 0 | 100 | 21 | 7.5 | RT | Yes |
| 2.5 | 100 | 0 | 100 | 21 | 8.5 | RT | Yes |
| 2.5 | 25 | 50 | 75 | 21 | 6.5 | RT | No |
| 2.5 | 25 | 50 | 75 | 21 | 7.5 | RT | Yes |
| 2.5 | 25 | 50 | 75 | 21 | 8.3 | RT | Yes |
| 2.5 | 100 | 50 | 75 | 21 | 6.5 | RT | Yes |
| 2.5 | 100 | 50 | 75 | 21 | 7.5 | RT | Yes |
| 2.5 | 100 | 50 | 75 | 21 | 8.4 | RT | Yes |
| 2.5 | 25 | 0 | 100 | 21 | 6.3 | 30° C. | No |
| 2.5 | 25 | 0 | 100 | 21 | 8.2 | 30° C. | Yes |
| 2.5 | 25 | 0 | 100 | 21 | 7.4 | 30° C. | Yes |
| 2.5 | 100 | 0 | 100 | 21 | 6.5 | 30° C. | Yes |
| 2.5 | 100 | 0 | 100 | 21 | 7.5 | 30° C. | Yes |
| 2.5 | 100 | 0 | 100 | 21 | 8.5 | 30° C. | Yes |
| 2.5 | 25 | 50 | 75 | 21 | 6.5 | 30° C. | No |
| 2.5 | 25 | 50 | 75 | 21 | 7.5 | 30° C. | Yes |
| 2.5 | 25 | 50 | 75 | 21 | 8.3 | 30° C. | Yes |
| 2.5 | 100 | 50 | 75 | 21 | 6.5 | 30° C. | Yes |
| 2.5 | 100 | 50 | 75 | 21 | 7.5 | 30° C. | Yes |
| 2.5 | 100 | 50 | 75 | 21 | 8.4 | 30° C. | Yes |

In another experiment, a stock solution of sodium acetate was prepared by dissolving 0.8203 g of sodium acetate in 10 mL of sterile water. A stock solution of A0^{Lys-Nε-Arg}A21^{Gly}B29^{Lys-Nε-Arg}-insulin was prepared by dissolving 0.003 g of A0^{Lys-Nε-Arg}A21^{Gly}B29^{Lys-Nε-Arg}-insulin in 0.63 mL of stock solution A. The solution was cloudy and the pH was approximately 7.1. pH was adjusted to approximately 3.7 to clear the solution.

Crystallization was set up by first combining the A0^{Lys-Nε-Arg}A21^{Gly}B29^{Lys-Nε-Arg}-insulin with zinc oxide, adding stock solution A, sodium chloride and/or sodium acetate stock solution. pH of the solution was kept below 4. Sodium phosphate stock solution was then added, and a precipitate was formed. The final pH was adjusted to between 6.5 and 9.5. Each sample was then split into three portions. One sample was incubated at 5° C., one at 30° C. and the other sample was left at room temperature.

The tested conditions and observations are shown in Table 11. All concentrations are nominal.

TABLE 11

| A0^{Lys-Nε-Arg}A21^{Gly}B29^{Lys-Nε-Arg}- | | | | | | | |
|---|---|---|---|---|---|---|---|
| insulin | Zinc | NaCl | NaOAc | Na₂PO₄ | | | Crystals |
| (mg/mL) | (mcg/mL) | (mM) | (mM) | (mM) | pH | Temp | Observed |
| 2.4 | 25 | 0 | 100 | 21 | 6.5 | 5° C. | No |
| 2.4 | 25 | 0 | 100 | 21 | 7.5 | 5° C. | No |
| 2.4 | 25 | 0 | 100 | 21 | 8.4 | 5° C. | Yes |
| 2.4 | 100 | 0 | 100 | 21 | 6.3 | 5° C. | No |
| 2.4 | 100 | 0 | 100 | 21 | 7.4 | 5° C. | No |
| 2.4 | 100 | 0 | 100 | 21 | 8.6 | 5° C. | Yes |
| 2.4 | 25 | 50 | 75 | 21 | 6.6 | 5° C. | No |
| 2.4 | 25 | 50 | 75 | 21 | 7.4 | 5° C. | No |
| 2.4 | 25 | 50 | 75 | 21 | 8.4 | 5° C. | Yes |
| 2.4 | 100 | 50 | 75 | 21 | 6.5 | 5° C. | No |
| 2.4 | 100 | 50 | 75 | 21 | 7.5 | 5° C. | No |
| 2.4 | 100 | 50 | 75 | 21 | 8.3 | 5° C. | No |
| 2.4 | 25 | 0 | 100 | 21 | 6.5 | RT | No |
| 2.4 | 25 | 0 | 100 | 21 | 7.5 | RT | Yes |
| 2.4 | 25 | 0 | 100 | 21 | 8.4 | RT | Yes |
| 2.4 | 100 | 0 | 100 | 21 | 6.3 | RT | No |
| 2.4 | 100 | 0 | 100 | 21 | 7.4 | RT | No |
| 2.4 | 100 | 0 | 100 | 21 | 8.6 | RT | No |
| 2.4 | 25 | 50 | 75 | 21 | 6.6 | RT | No |
| 2.4 | 25 | 50 | 75 | 21 | 7.4 | RT | Yes |
| 2.4 | 25 | 50 | 75 | 21 | 8.4 | RT | Yes |
| 2.4 | 100 | 50 | 75 | 21 | 6.5 | RT | No |
| 2.4 | 100 | 50 | 75 | 21 | 7.5 | RT | No |
| 2.4 | 100 | 50 | 75 | 21 | 8.3 | RT | Yes |
| 2.4 | 25 | 0 | 100 | 21 | 6.5 | 30° C. | No |
| 2.4 | 25 | 0 | 100 | 21 | 7.5 | 30° C. | Yes |
| 2.4 | 25 | 0 | 100 | 21 | 8.4 | 30° C. | Yes |
| 2.4 | 100 | 0 | 100 | 21 | 6.3 | 30° C. | No |
| 2.4 | 100 | 0 | 100 | 21 | 7.4 | 30° C. | Yes |
| 2.4 | 100 | 0 | 100 | 21 | 8.6 | 30° C. | No |
| 2.4 | 25 | 50 | 75 | 21 | 6.6 | 30° C. | No |
| 2.4 | 25 | 50 | 75 | 21 | 7.4 | 30° C. | Yes |
| 2.4 | 25 | 50 | 75 | 21 | 8.4 | 30° C. | No |
| 2.4 | 100 | 50 | 75 | 21 | 6.5 | 30° C. | No |
| 2.4 | 100 | 50 | 75 | 21 | 7.5 | 30° C. | Yes |
| 2.4 | 100 | 50 | 75 | 21 | 8.3 | 30° C. | Yes |

In another experiment, a zinc oxide stock solution was prepared by diluting 1.0 mL of a 10 mg/mL zinc oxide solution with 1.0 mL of sterile water. The final zinc oxide concentration was 5 mg/mL.

A stock solution of A0^{Lys-Nε-Arg}A21^{Gly}B29^{Lys-Nε-Arg}-insulin was prepared by dissolving 0.00221 g of A0^{Lys-Nε-Arg}A21B29^{Lys-Nε-Arg}-insulin in 0.43 mL of sterile water. The solution was almost clear and the pH was checked to be approximately 3.7. pH was adjusted to approximately 3.0 to clear the solution.

Crystallization was set up by first combining the A0^{Lys-Nε-Arg}A21^{Gly}B29^{Lys-Nε-Arg}-insulin with zinc oxide, and either sodium chloride or sodium citrate or sodium acetate stock solution. pH of the solution was kept below around 3. Sodium phosphate stock solution was then added, and a precipitate was formed. The final pH was adjusted to between 6.5 and 8.5. Each sample was left at room temperature. The tested conditions and observations are shown in Table 12. All concentrations are nominal.

TABLE 12

| A0^{Lys-Nε-Arg}A21^{Gly}B29^{Lys-Nε-Arg}- | | | Na | | | |
|---|---|---|---|---|---|---|
| insulin | Zinc | NaCl | Citrate | NaOAc | | Crystals |
| (mg/mL) | (mcg/mL) | (mM) | (mM) | (mM) | pH | Observed |
| 2.6 | 300 | 0 | 0 | 0 | 6.7 | No |
| 2.6 | 300 | 0 | 0 | 0 | 8.4 | No |
| 2.6 | 300 | 100 | 0 | 0 | 8.3 | No |
| 2.6 | 300 | 100 | 0 | 0 | 6.6 | No |
| 2.6 | 300 | 0 | 100 | 0 | 6.6 | Yes |
| 2.6 | 300 | 0 | 100 | 0 | 8.2 | No |
| 2.6 | 300 | 0 | 0 | 100 | 6.5 | Yes |
| 2.6 | 300 | 0 | 0 | 100 | 8.6 | No |

### EXAMPLE 23

### A0^{Lys-Nε-Arg}A21^{Gly}B29^{Lys-Nε-Arg}-Insulin Protamine-Zinc Crystals

A stock solution A is prepared by dissolving 16.1 g of synthetic glycerin, 0.73 g of phenol and 1.6 mL of m-cresol in approximately 350 mL of sterile water. After dissolution, sterile water is added to a final solution weight of 503 g. A protamine sulfate stock solution is prepared by dissolving 0.0366 g of protamine sulfate in 10 mL of sterile water.

An A0^{Lys-Nε-Arg}A21^{Gly}B29^{Lys-Nε-Arg}-insulin stock solution is prepared by dissolving 0.0121 g of A0^{Lys-Nε-Arg}A21^{Gly}B29^{Lys-Nε-Arg}-insulin in 1.28 mL of stock solution A. A zinc oxide stock solution is prepared by diluting 1 mL of a 25 mg/mL zinc oxide solution to a final volume of 25 mL, to obtain a final zinc oxide concentration of 1 mg/mL. A sodium phosphate stock solution is prepared by dissolving 0.0577 g of dibasic sodium phoshphate in 15 mL of sterile water. A sodium chloride stock solution is prepared by dissolving 1.1607 g of sodium chloride in 10 mL of sterile water.

A0^{Lys-Nε-Arg}A21^{Gly}B29^{Lys-Nε-Arg}-insulin, zinc oxide, protamine sulfate, sodium chloride and stock solution A are combined to a final volume of 0.1 mL to yield different conditions. 0.1 mL of sodium phosphate stock solution is added, and a precipitate is formed. The final pH is adjusted to between 7.4 and 9.3.

Each sample is then split into two halves. One sample is incubated at 30° C. and the other sample is left at room temperature. The conditions tested are shown in Table 13, and crystals are observed.

TABLE 13

| | Prota- | | | | |
|---|---|---|---|---|---|
| | mine | | | | |
| A0^{Lys-Nε-Arg}A21^{Gly}B29^{Lys-Nε-Arg}- | sulphate | Zinc | Nacl | | |
| insulin (mg/ml) | (mg/ml) | Mcg/ml | (mM) | pH | Temp |
| 3.5 | 0 | 25 | 0 | 7.4 | RT |
| 3.5 | 0 | 25 | 100 | 7.5 | RT |
| 3.5 | 0 | 25 | 0 | 8.5 | RT |
| 3.5 | 0 | 25 | 100 | 8.5 | RT |
| 3.5 | 0 | 25 | 0 | 9.3 | RT |
| 3.5 | 0 | 25 | 100 | 9.2 | RT |
| 3.5 | 0.37 | 25 | 100 | 7.4 | RT |
| 3.5 | 0.37 | 25 | 100 | 8.5 | RT |
| 3.5 | 0.37 | 25 | 100 | 9.2 | RT |
| 3.5 | 0 | 25 | 0 | 7.4 | 30° C. |
| 3.5 | 0 | 25 | 100 | 7.5 | 30° C. |
| 3.5 | 0 | 25 | 0 | 8.5 | 30° C. |
| 3.5 | 0 | 25 | 100 | 8.5 | 30° C. |
| 3.5 | 0 | 25 | 0 | 9.3 | 30° C. |
| 3.5 | 0 | 25 | 100 | 9.2 | 30° C. |
| 3.5 | 0.37 | 25 | 100 | 7.4 | 30° C. |
| 3.5 | 0.37 | 25 | 100 | 8.5 | 30° C. |
| 3.5 | 0.37 | 25 | 100 | 9.2 | 30° C. |

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

All patents, patent applications, articles, books, and other publications cited herein are incorporated by reference in their entireties.

1. An insulin molecule having
(a) an A-chain of Formula I,
A-1 A0 A1 A2 A3 A4 A5 A6 A7 A8 A9 A10
Xaa-Xaa-Gly-Ile-Val-Glu-Gln-Cys-Cys-Thr-Ser-Ile-
A11 A12 A13 A14 A15 A16 A17 A18 A19 A20 A21
Cys-Ser-Leu-Tyr-Gln-Leu-Glu-Asn-Tyr-Cys-Xaa,
wherein the amino acid sequence of Formula I is set forth in Seq. ID No. 1, and
(b) a B-chain of Formula II,
B-1 B0 B1 B2 B3 B4 B5 B6 B7 B8 B9 B10
Xaa-Xaa-Phe-Val-Asn-Gln-His-Leu-Cys-Gly-Ser-His-
B11 B12 B13 B14 B15 B16 B17 B18 B19 B20 B21 B22
Leu-Val-Glu-Ala-Leu-Tyr-Leu-Val-Cys-Gly-Glu-Arg-
B23 B24 B25 B26 B27 B28 B29 B30
Gly-Phe-Phe-Tyr-Thr-Xaa-Xaa-Xaa,
wherein the amino acid sequence of Formula II is set forth in Seq. ID No. 2,
wherein Xaa at position A-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position A0 is Arg, derivatized Arg, homoarginine, desamino homoarginine, desaminoarginine, Lys, derivatized Lys, desaminolysine, alpha guanidino homoarginine, or alpha methyl arginine;
Xaa at position A21 is a genetically encodable amino acid;
Xaa at position B-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position B0 is Arg, derivatized Arg, homoarginine, desamino homoarginine, desaminoarginine, Lys, derivatized Lys, desaminolysine, alpha guanidino homoarginine, alpha methyl arginine or is absent;
Xaa at position B28 is Lys or Pro;
Xaa at position B29 is Lys or Pro;
Xaa at position B30 is Thr, Ala or is absent;
one of Xaa at position B28 or Xaa at position B29 is Lys;
Xaa at position B28 and Xaa at position B29 are not both Lys; and
the ε-amino group of Lys at position B28 or B29 is covalently bound to the α-carboxyl group of a positively charged amino acid to form a Lys-Nε-amino acid derivative.

2. The insulin molecule of claim 1, wherein the ε-amino group of Lys at position B28 or B29 is covalently bound to the α-carboxyl group of Arg to form Lys-Nε-Arg.

3. The insulin molecule of claim 1, wherein the ε-amino group of Lys at position B28 or B29 is covalently bound to the α-carboxyl group of Lys to form Lys-Nε-Lys.

4. The insulin molecule of claim 1, wherein Xaa at position A-1 and Xaa at position B-1 are absent.

5. The insulin molecule of claim 1, wherein Xaa at position B-1 and Xaa at position B0 are absent.

6. The insulin molecule of claim 1, wherein Xaa at position A-1, Xaa at position B-1 and Xaa at position B0 are absent.

7. (Cancelled)

8. The insulin molecule of claim 6, wherein Xaa at position A0 is Arg.

9. The insulin molecule of claim 4,
wherein Xaa at position A0 is Arg; and
Xaa at position B0 is Arg.

10. (Cancelled)

11. A composition comprising the insulin molecule of claim 1.

12. (Cancelled)

13. The composition of claim 11, further comprising one or more pharmaceutically acceptable excipients.

14. The composition of claim 13, further comprising a divalent metal cation.

15. The composition of claim 14, wherein the divalent metal cation is zinc.

16. The composition of claim 11, further comprising human insulin.

17. The composition of claim 11, further comprising a rapid-acting insulin analog.

18. (Cancelled)

19. A microcrystal comprising the insulin molecule of claim 11 and a divalent metal cation, wherein the microcrystal does not contain protamine.

20. The microcrystal of claim 19, wherein the divalent metal cation is zinc.

21. (Cancelled)

22. (Cancelled)

23. A process for preparing the microcrystal of claim 19, comprising contacting ingredients comprising the insulin molecule, and a divalent metal cation in aqueous solvent at a pH that permits formation of hexamers of the insulin molecule.

24. The process of claim 23, wherein the divalent metal cation is zinc.

25. (Canceled)

26. (Cancelled)

27. A method of making an insulin molecule, the method comprising:
(a) acylating each free amino group of an insulin template with a protected amino acid or a protected amino acid derivative to form an acylated insulin molecule;
(b) purifying the acylated insulin molecule;
(c) removing the protecting group from each protected amino acid or protected amino acid derivative to form a deprotected acylated insulin molecule; and
(d) purifying the deprotected acylated insulin molecule.

28. (Canceled)

29. (Canceled)

30. (Canceled)

31. (Canceled)

32. (Canceled)

33. A method of treating hyperglycemia, the method comprising administering the composition of claim 11 to a subject in an amount sufficient to regulate blood glucose concentration in the subject.

34. (Canceled)

35. (Canceled)

36. An insulin molecule having
(a) an A-chain of Formula I,
A-1 A0 A1 A2 A3 A4 A5 A6 A7 A8 A9 A10
Xaa-Xaa-Gly-Ile-Val-Glu-Gln-Cys-Cys-Thr-Ser-Ile-
A11 A12 A13 A14 A15 A16 A17 A18 A19 A20 A21
Cys-Ser-Leu-Tyr-Gln-Leu-Glu-Asn-Tyr-Cys-Xaa,
wherein the amino acid sequence of Formula I is set forth in Seq. ID No. 1, and
(b) a B-chain of Formula II,
B-1 B0 B1 B2 B3 B4 B5 B6 B7 B8 B9 B10
Xaa-Xaa-Phe-Val-Asn-Gln-His-Leu-Cys-Gly-Ser-His-
B11 B12 B13 B14 B15 B16 B17 B18 B19 B20 B21 B22
Leu-Val-Glu-Ala-Leu-Tyr-Leu-Val-Cys-Gly-Glu-Arg-
B23 B24 B25 B26 B27 B28 B29 B30
Gly-Phe-Phe-Tyr-Thr-Xaa-Xaa-Xaa,
wherein the amino acid sequence of Formula II is set forth in Seq. ID No. 2,
wherein Xaa at position A-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position A0 is Arg, derivatized Arg, homoarginine, desamino homoarginine, desaminoarginine, Lys, derivatized Lys, desaminolysine, alpha guanidino homoarginine, alpha methyl arginine;
Xaa at position A21 is a genetically encodable amino acid;
Xaa at position B-1 is Arg, derivatized Arg, homoarginine, desamino homoarginine, desaminoarginine, Lys, derivatized Lys, desaminolysine, alpha guanidino homoarginine, alpha methyl arginine, or is absent;
Xaa at position B0 is Arg, derivatized Arg, homoarginine, desamino homoarginine, desaminoarginine, Lys, derivatized Lys, desaminolysine, alpha guanidino homoarginine, alpha methyl arginine;
Xaa at position B28 is Lys or Pro;
Xaa at position B29 is Lys or Pro;
Xaa at position B30 is Thr, Ala or is absent;
one of Xaa at position B28 or Xaa at position B29 is Lys; and
Xaa at position B28 and Xaa at position B29 are not both Lys.

37. The insulin molecule of claim 36, wherein
Xaa at position A-1 is absent; and
Xaa at position B-1 is absent.

38. The insulin molecule of claim 37, wherein
Xaa at position A0 is Arg or Lys; and
Xaa at position B0 is Arg or Lys.

39. The insulin molecule of claim 38, wherein
Xaa at position A0 is Arg; and
Xaa at position B0 is Arg.

40. A microcrystal comprising the insulin molecule of claim 36 and a divalent cation.

41. The microcrystal of claim 40, wherein the divalent metal cation is zinc.

42. The microcrystal of claim 40, further comprising protamine.

43. A composition comprising the insulin molecule of claim 36.

44. (Canceled)

45. (Canceled)

46. (Canceled)

47. The composition of claim 43, further comprising one or more pharmaceutically acceptable excipients.

48. (Canceled)

49. (Canceled)

50. A method of treating hyperglycemia, the method comprising administering the composition of claim 43 to a subject in an amount sufficient to regulate blood glucose concentration in the subject.

51. (Canceled)

52. (Canceled)

53. (Canceled)


SEQUENCE LISTING (4 sequences)

SEQ ID NO: 1 - protein, 23 aa, Homo sapiens
  MISC_FEATURE (1)..(1): Xaa = Arg, derivatized Arg, homoarginine, desamino homoarginine, desaminoarginine, Lys, derivatized Lys, desaminolysine, alpha guanidino homoarginine, alpha methyl arginine, or is absent
XXGIVEQCCTSICSLYQLENYCX

SEQ ID NO: 2 - protein, 32 aa, Homo sapiens
  MISC_FEATURE (1)..(1): Xaa = Arg, derivatized arg, homoarginine, desamino homoarginine, desaminoarginine, Lys, derivatized Lys, desaminolysine, alpha guanidino homoarginine, alpha methyl arginine, or is absent
XXFVNQHLCGSHLVEALYLVCGERGFFYTXXX

SEQ ID NO: 3 - protein, 21 aa, Homo sapiens
GIVEQCCTSICSLYQLENYCN

SEQ ID NO: 4 - protein, 30 aa, Homo sapiens
FVNQHLCGSHLVEALYLVCGERGFFYTPKT